(19) United States
(12) Patent Application Publication
(10) Pub. No.: US 2026/0096734 A1
(43) Pub. Date: Apr. 9, 2026

(54) Multiparameter Cuffless Blood Pressure Monitoring System

(71) Applicant: iRhythm Technologies, Inc., San Francisco, CA (US)

(72) Inventor: Steven Robert Keyes, San Francisco, CA (US)

(73) Assignee: iRhythm Technologies, Inc., San Francisco, CA (US)

(21) Appl. No.: 19/341,842

(22) Filed: Sep. 26, 2025

Related U.S. Application Data
(60) Provisional application No. 63/703,665, filed on Oct. 4, 2024.

(51) Int. Cl. A61B 5/021; A61B 5/024; A61B 5/026; A61B 5/318

(52) U.S. Cl. CPC A61B 5/02125; A61B 5/02416; A61B 5/0261; A61B 5/318; A61B 2560/0223; A61B 2562/06

(57) ABSTRACT

Techniques for cuffless blood pressure monitoring with multiparameter correction are described and are implementable to reduce measurement inaccuracies in wearable cuffless blood pressure devices. In an example, a wearable device includes a sensor arrangement to collect physiological timing data indicative of a pulse propagation time along a cardiovascular pathway and a correction sensor that is configured to measure a correction parameter that impacts the pulse propagation time independent of a corresponding change to blood pressure, such as body temperature, skin temperature, perfusion index, hydration level, or muscle activation. A processor of the wearable device is configured to process the physiological timing data to determine the pulse propagation time and generate a blood pressure measurement based on the pulse propagation time and measurements of the correction parameter. Accordingly, the techniques described herein generate blood pressure measurements that account for physiological variables that have an independent impact on pulse propagation characteristics.

### RELATED APPLICATIONS

This application claims priority to U.S. Application No. 63/703,665, titled Cuffless Blood Pressure Monitor with Additional Sensors for Correction, filed October 4, 2024, which is hereby incorporated by reference in its entirety.

### BACKGROUND

Blood pressure measurements can provide important insights into an individual’s cardiovascular health. Traditionally, blood pressure has been measured using inflatable cuffs that temporarily occlude blood flow in an artery and detect pressure oscillations or sounds as the cuff deflates which can be correlated to blood pressure values. While these devices can provide accurate measurements, conventional cuff-based devices are often not suitable for continuous monitoring in ambulatory scenarios due to an obstructive nature of inflation/deflation cycles, bulky form factors that restrict wearability, and intermittent nature of measurements that create gaps in data collection.

### BRIEF DESCRIPTION OF THE DRAWINGS

FIG. 1 is a block diagram of a nonlimiting example of an environment that is operable to employ multiparameter cuffless blood pressure monitoring systems and techniques as described herein.

FIG. 2 depicts a nonlimiting example of a monitoring device.

FIG. 3 depicts a nonlimiting system in an example implementation of multiparameter cuffless blood pressure monitoring showing operation of a blood pressure monitoring system in more detail.

FIG. 4 depicts a nonlimiting example of multiparameter cuffless blood pressure monitoring in which a wearable device generates a blood pressure measurement based on timing data and one or more measured correction parameters.

FIG. 5 depicts a nonlimiting example of multiparameter cuffless blood pressure monitoring in which a temperature sensor is used to collect skin temperature data as a correction parameter.

FIG. 6 depicts an additional nonlimiting example of multiparameter cuffless blood pressure monitoring in which a temperature sensor is used to collect skin temperature data as a correction parameter.

FIG. 7 depicts a nonlimiting example of multiparameter cuffless blood pressure monitoring in which a heat flux sensor is used to collect body temperature data as a correction parameter.

FIG. 8 depicts a nonlimiting example of multiparameter cuffless blood pressure monitoring in which a PPG sensor is used to collect perfusion index data as a correction parameter.

FIG. 9 depicts a nonlimiting example of multiparameter cuffless blood pressure monitoring in which a bioimpedance sensor is used to collect hydration level data as a correction parameter.

FIG. 10 depicts a nonlimiting example of multiparameter cuffless blood pressure monitoring in which an EMG sensor is used to collect muscle activation data as a correction parameter.

FIG. 11 depicts a nonlimiting example of multiparameter cuffless blood pressure monitoring in which multiple correction parameters are used to generate a blood pressure measurement.

FIG. 12 depicts a nonlimiting example of multiparameter cuffless blood pressure monitoring in which a user interface for a blood pressure monitoring scenario is shown.

FIG. 13 depicts a flow diagram depicting an algorithm as a step-by-step procedure in an example implementation to generate a blood pressure using multiparameter cuffless blood pressure monitoring techniques.

### DETAILED DESCRIPTION

Cuff-based blood pressure monitors, while accurate and useful for single measurements, often face practical limitations that prevent use of such devices for continuous health monitoring applications. Accordingly, development of cuffless blood pressure monitoring technologies has emerged as an alternative to provide continuous, non-invasive measurements. Cuffless systems aim to utilize physiological signals to estimate blood pressure rather than relying on mechanical occlusion of blood vessels. For instance, pulse wave characteristics, such as pulse transit time (“PTT”) or pulse arrival time (“PAT”), can be used to estimate blood pressure based on a relationship between arterial stiffness and pulse wave velocity.

However, cuffless blood pressure monitoring systems may experience phenomena such as calibration “drift” and become inaccurate over time and thus require frequent recalibration against traditional cuff-based measurements to maintain accuracy. For instance, conventional cuffless systems are susceptible to various physiological and environmental factors as well as variations between individuals that can affect pulse wave characteristics independent of blood pressure and thus decrease measurement reliability. These limitations may compromise long-term accuracy and offset advantages of conventional cuffless monitoring devices.

Accordingly, a multiparameter cuffless blood pressure monitoring system is described to address these limitations by measuring and accounting for physiological and/or environmental factors that can cause calibration drift. By way of example, the system includes a wearable device with a sensor arrangement that is configured to collect physiological timing data that is indicative of pulse wave characteristics such as a pulse propagation time, e.g., pulse transit time (“PTT”) and/or pulse arrival time (“PAT”), along a cardiovascular pathway between proximal and distal measurement points.

In this example, the sensor arrangement includes a proximal sensor, such as an electrocardiogram (“ECG”) sensor or bioimpedance sensor, positioned to detect a first cardiovascular signal, and a distal sensor, such as a photoplethysmography (“PPG”) sensor, positioned to detect a corresponding second cardiovascular signal at a distal location. The wearable device leverages a processor to determine a pulse propagation time by detecting a temporal difference between the first cardiovascular signal and the second cardiovascular signal, which represents a duration for a pulse wave to travel along the cardiovascular pathway. The pulse propagation time may be correlated to blood pressure through established relationships between arterial stiffness and pulse wave velocity. For instance, a relatively high blood pressure may correspond to increased arterial stiffness and faster pulse wave propagation, resulting in shorter transit times.

However, environmental and/or physiological factors such as body temperature, skin temperature, hydration, and/or muscle activation can affect pulse wave velocity partially or wholly independent of corresponding changes to blood pressure, which can cause calibration drift and/or inaccurate measurements using conventional systems. To account for these factors, the wearable device further includes one or more correction sensors configured to measure correction parameters that impact pulse propagation time independent of corresponding changes to blood pressure. Accordingly, the wearable device can generate accurate blood pressure measurements based on the physiological timing data as well as measurements of the correction parameter.

In various embodiments, the correction sensors may include one or more temperature sensors and/or heat flux sensors configured to measure body temperature and/or skin temperature, such as to account for effects of vasomotion (e.g., vasodilation or vasoconstriction) and/or systemic cardiovascular changes. Additionally or alternatively, the correction sensors may include bioimpedance sensors configured to measure hydration levels such as to account for arterial stiffness variations, PPG sensors to calculate a perfusion index based on a ratio of pulsatile to non-pulsatile blood flow, and/or electromyography (“EMG”) sensors for measuring muscle activation by detecting electrical signals from smooth muscle tissue.

The processor is configured to process the physiological timing data to determine pulse propagation time, such as PTT or PAT, and generate blood pressure measurements based on both the pulse propagation time and measurements of the one or more correction parameters. For instance, the processor can generate an initial blood pressure measurement based on the pulse propagation time and apply a correction factor based on measurements of the one or more correction parameters to generate an adjusted blood pressure measurement.

In various examples, the system continuously integrates correction factors into blood pressure calculations, selectively activates corrections based on threshold conditions such as to optimize power consumption, and/or performs post-processing analysis of collected data based on the correction factors. The system further is operable to implement weighting schemes for multiple correction parameters, utilize various blood pressure measurement algorithms, and/or leverage machine learning models trained to calibrate blood pressure readings based on the various correction parameters. This is by way of example and not limitation, and the system is operable and implementable in a variety of ways to generate blood pressure measurements based on timing data and measurements of correction parameters.

In this way, the techniques, devices, and components described herein overcome the limitations of conventional systems by preventing calibration drift in cuffless blood pressure monitoring. For instance, the multiparameter cuffless blood pressure monitoring system can account for temperature-induced vasoconstriction that affects peripheral pulse wave velocity, hydration-related changes in arterial stiffness, perfusion variations that influence peripheral circulation, smooth muscle activation that affects arterial wall properties, and so forth. Thus, the system maintains accuracy over extended periods without manual recalibration to support continuous blood pressure monitoring and generation of accurate cardiovascular health insights over an extended wear period.

In some aspects, the techniques described herein relate to a wearable device for cuffless blood pressure monitoring including: a sensor arrangement to collect physiological timing data indicative of a pulse propagation time along a cardiovascular pathway; a correction sensor configured to measure a correction parameter that impacts the pulse propagation time independent of a corresponding change to blood pressure; and a processor configured to: process the physiological timing data to determine the pulse propagation time; and present a blood pressure measurement generated based on the pulse propagation time and measurements of the correction parameter.

In some aspects, the techniques described herein relate to a wearable device, wherein the sensor arrangement includes a proximal sensor positioned at a proximal location along the cardiovascular pathway configured to detect a first cardiovascular signal as part of the physiological timing data, and a distal sensor positioned at a distal location along the cardiovascular pathway configured to detect a second cardiovascular signal as part of the physiological timing data that corresponds to the first cardiovascular signal.

In some aspects, the techniques described herein relate to a wearable device, wherein the proximal sensor includes one or more of a proximal electrocardiogram sensor, a proximal bioimpedance sensor, or a proximal photoplethysmography sensor, the distal sensor includes one or more of a distal photoplethysmography sensor, a distal pressure sensor, or a distal bioimpedance sensor, and the pulse propagation time includes one or more of a pulse transit time (PTT) or a pulse arrival time (PAT).

In some aspects, the techniques described herein relate to a wearable device, wherein the correction sensor includes one or more of a temperature sensor or a heat flux sensor configured to measure a skin temperature adjacent to the sensor arrangement as the correction parameter, and the processor is further configured to generate the blood pressure measurement based on the skin temperature to account for an impact of vasomotion on the pulse propagation time.

In some aspects, the techniques described herein relate to a wearable device, wherein the correction sensor includes one or more of a temperature sensor or a heat flux sensor configured to measure a body temperature as the correction parameter, and the processor is further configured to generate the blood pressure measurement based on the body temperature to account for systemic cardiovascular effects on the pulse propagation time.

In some aspects, the techniques described herein relate to a wearable device, wherein the correction sensor includes a bioimpedance sensor configured to measure a hydration level as the correction parameter, and the processor is further configured to generate the blood pressure measurement based on the hydration level to account for an impact of arterial stiffness on the pulse propagation time.

In some aspects, the techniques described herein relate to a wearable device, wherein the correction sensor includes a photoplethysmography (PPG) sensor configured to collect PPG data, the processor further configured to: calculate a perfusion index as the correction parameter by determining a ratio of pulsatile blood flow to non-pulsatile blood flow based on the PPG data; and generate the blood pressure measurement based on the perfusion index to account for an impact of peripheral circulation on the pulse propagation time.

In some aspects, the techniques described herein relate to a wearable device, wherein the correction sensor includes an electromyography sensor configured to measure muscle activation as the correction parameter by detecting electrical signals associated with smooth muscle tissue, the processor further configured to generate the blood pressure measurement based on the muscle activation to account for arterial wall contractility on the pulse propagation time.

In some aspects, the techniques described herein relate to a wearable device, wherein the processor is further configured to generate the blood pressure measurement by calculating an initial blood pressure measurement based on the pulse propagation time and applying a correction factor to the initial blood pressure measurement based on the correction parameter responsive to a detection that the correction parameter exceeds a threshold.

In some aspects, the techniques described herein relate to a wearable device, wherein the processor is further configured to determine a systolic blood pressure value and a diastolic blood pressure value to include as part of the blood pressure measurement based on one or more characteristics of the pulse propagation time and the correction parameter.

In some aspects, the techniques described herein relate to a method implemented by a processing device, the method including: receiving physiological timing data indicative of a pulse propagation time along a cardiovascular pathway and measurements of a correction parameter that impacts the pulse propagation time independent of a corresponding change to blood pressure; processing the physiological timing data to determine the pulse propagation time based on temporal differences between corresponding points in the physiological timing data; generating a blood pressure measurement based on the pulse propagation time and the measurements of the correction parameter; and outputting the blood pressure measurement.

In some aspects, the techniques described herein relate to a method, wherein the correction parameter includes at least one of body temperature, skin temperature, perfusion index, hydration level, or muscle activation.

In some aspects, the techniques described herein relate to a method, wherein the physiological timing data includes a first physiological signal and a second physiological signal, and the processing the physiological timing data includes determining one or more of a pulse transit time (PTT) or a pulse arrival time (PAT) as the pulse propagation time based on the first physiological signal and the second physiological signal.

In some aspects, the techniques described herein relate to a method, wherein the first physiological signal includes an electrocardiogram (ECG) signal and the second physiological signal includes a photoplethysmography (PPG) signal.

In some aspects, the techniques described herein relate to a method, wherein generating the blood pressure measurement includes generating an initial blood pressure measurement based on the pulse propagation time and adjusting the initial blood pressure measurement based on the measurements of the correction parameter.

In some aspects, the techniques described herein relate to a method, wherein generating the blood pressure measurement includes applying a correction factor to the blood pressure measurement based on the measurements of the correction parameter responsive to a detection that the correction parameter exceeds a threshold.

In some aspects, the techniques described herein relate to a system for cuffless blood pressure monitoring including: a sensor arrangement to collect physiological timing data indicative of a pulse propagation time along a cardiovascular pathway; one or more correction sensors configured to measure one or more correction parameters that impact the pulse propagation time independent of corresponding changes to blood pressure; and a processor configured to: process the physiological timing data to determine the pulse propagation time; generate a blood pressure measurement based on the pulse propagation time and measurements of the one or more correction parameters; and cause output of the blood pressure measurement.

In some aspects, the techniques described herein relate to a system, wherein the sensor arrangement includes a first sensor configured to measure a first physiological signal via contact with a skin surface of a user and a second sensor configured to measure a second physiological signal via contact with the skin surface of the user, and the processor is further configured to generate one or more of a pulse transit time (PTT) or a pulse arrival time (PAT) as the pulse propagation time based on the first physiological signal and the second physiological signal.

In some aspects, the techniques described herein relate to a system, wherein the measurements of the one or more correction parameters include measurements of at least two of body temperature, skin temperature, perfusion index, hydration level, or muscle activation, and the processor is further configured to implement a weighting scheme to apply weights to the measurements of the one or more correction parameters to generate the blood pressure measurement.

In some aspects, the techniques described herein relate to a system, wherein the processor is configured to implement one or more of a blood pressure calibration algorithm, a machine learning model trained to calibrate blood pressure readings based on correction parameters, or a calibration curve to generate the blood pressure measurement.

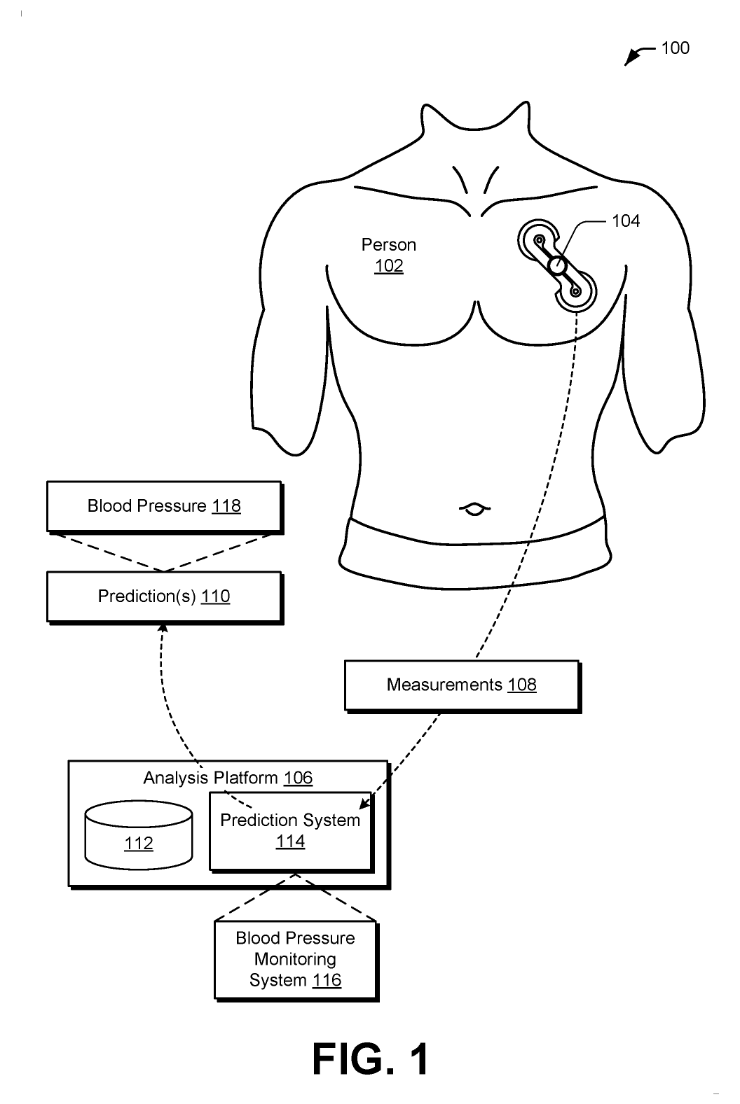

FIG. 1 is a block diagram of a nonlimiting example 100 of an environment that is operable to employ multiparameter cuffless blood pressure monitoring systems and techniques as described herein. The illustrated example 100 includes person 102, who is depicted wearing a monitoring device 104. The illustrated environment also includes an analysis platform 106. The analysis platform 106 may be connected to the monitoring device 104 via one or more wireless connections directly or via one or more wired and/or wireless connections and one or more intermediate devices, such as a computing device associated with the person 102, network routing devices and equipment, server devices, and/or the Internet, to name just a few.

The monitoring device 104 may be utilized to monitor one or more aspects of the person 102, such as to generate measurements 108. In some scenarios, for instance, the monitoring device 104 may be provided to record electrical activity of the person 102’s heart over an observation period, e.g., lasting some number of seconds or minutes, lasting multiple days, and so on. By way of example, the person 102 may have a magnitude of his or her heart’s electrical potential monitored over time to produce one or more electrocardiograms, which may be used to predict any of a variety of events. In at least one example, the monitoring device 104 is provided to record physiological timing data, e.g., data that is indicative of one or more pulse wave characteristics, and/or measurements of correction parameters that impact pulse wave characteristics. Alternatively or in addition, the monitoring device 104 may be used to output measurements 108 (e.g., a time sequence of measurements such as a time sequence of electric potential measurements), which may indicate an observation or be used to generate a prediction of one or more events.

In connection with the monitoring device, instructions may be provided to the person 102 that instruct the person 102 how to operate the monitoring device 104 and/or how to behave (e.g., sleep, perform activity) while wearing monitoring device 104. In one or more implementations, the instructions may be provided as part of a kit, e.g., written instructions. Alternately or additionally, the analysis platform 106 may cause the instructions to be communicated to and output (e.g., for display and/or audio output) via a computing device associated with the person 102. In one or more implementations, the analysis platform 106 may wait to provide these instructions for output after a predetermined amount of time of an observation period has lapsed (e.g., two days) while wearing the monitoring device 104 and/or based on patterns in the aspects of the person 102 being measured.

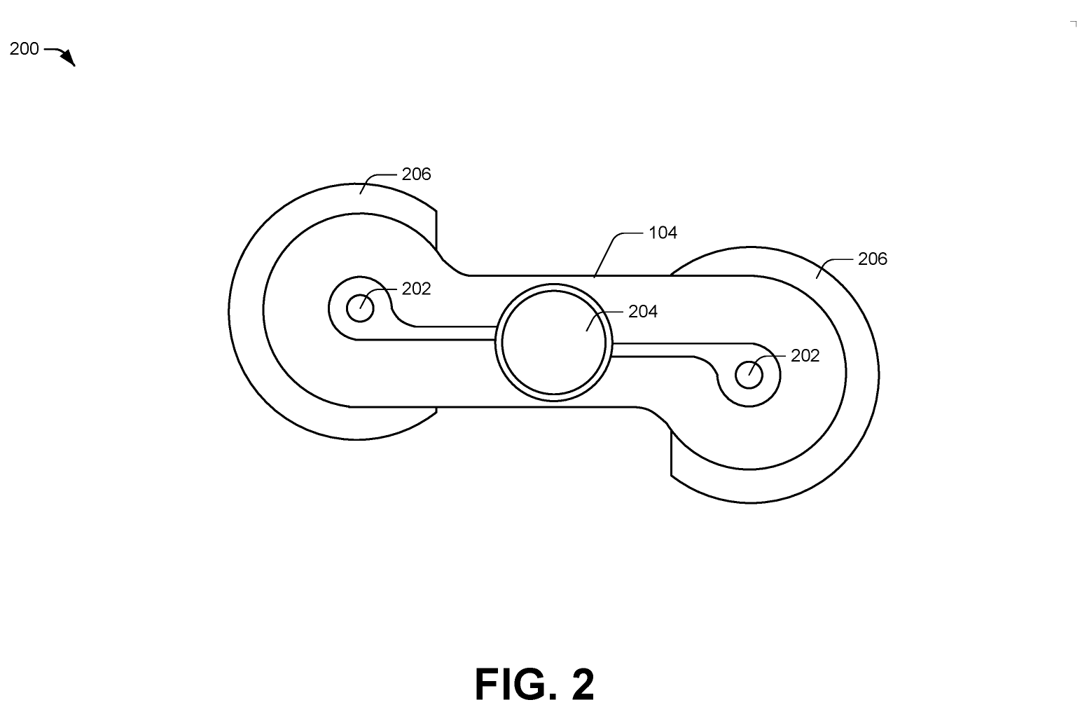

The monitoring device 104 may be configured in a variety of ways to monitor one or more aspects of the person 102. Moreover, the form factor depicted in FIGS. 1 and 2 is just one example form factor, and the form factor of the monitoring device 104 may differ in variations. It is to be appreciated that the monitoring device 104 may be configured with one or more sensors, examples of which include one or more of: a plurality of electrodes (e.g., that can be placed on the skin of the person), an accelerometer, and a pulse oximeter (e.g., to measure and record oxygen saturation (SpO2) and/or produce a photoplethysmogram of the person 102), to name just a few. Certainly, the monitoring device 104 may be configured with any of a variety of types of sensors without departing from the described techniques.

Although the monitoring device 104 may be configured in a similar manner as monitoring devices used for clinically monitoring patients, in one or more implementations, the monitoring device 104 may be configured differently than the devices used for monitoring and/or diagnosing patients clinically. By way of example, and not limitation, the monitoring device 104 may be configured as a ring, a watch, a patch, and/or a strap, to name just a few form factors. Alternatively or additionally, the monitoring device 104 may have a similar form factor as for clinical settings, but have different functionality, such as functionality that prevents a wearer from viewing the measurements 108.

In one or more implementations, the monitoring device 104 may be configured to offload measurements 108 during the course of the observation period. By way of example, the monitoring device 104 may offload the measurements 108 by transmitting them via a wired or wireless connection to an external computing device, e.g., at predetermined time intervals and/or responsive to establishing or reestablishing a connection with the computing device. In one or more implementations, the measurements 108 and/or other data from the monitoring device 104 may be compressed by the monitoring device 104 for wireless transmission, e.g., using one or more of a variety of data compression techniques. Compression of the sensor data in this way can reduce battery usage of the monitoring device 104 during the observation period and facilitate wear during assessments of physiological conditions, e.g., blood pressure related conditions.

To the extent that the monitoring device 104 may be configured to store the measurements 108 for an entirety of an observation period, in one or more implementations, the monitoring device 104 may be configured without wireless transmission means, e.g., without any antennae to transmit the measurements 108 wirelessly and without hardware or firmware to generate packets for such wireless transmission. Instead, the monitoring device 104 may be configured with hardware to communicate the measurements 108 via a physical, wired coupling. In such scenarios, the monitoring device 104 may be “plugged in” to extract the measurements 108 from the device’s storage.

Accordingly, the monitoring device 104 may be configured with one or more ports to enable wired transmission of the measurements 108 to an external computing device. Examples of such physical couplings may include micro universal serial bus (USB) connections, mini-USB connections, and USB-C connections, to name just a few. Although the monitoring device 104 may be configured for extraction of the measurements 108 via wired connections as discussed just above, in different scenarios, the monitoring device 104 may alternately or additionally be configured to offload the measurements 108 over one or more wireless connections.

Once the monitoring device 104 produces the measurements 108, the measurements 108 are provided to the analysis platform 106. As noted above, the measurements 108 may be communicated to the analysis platform 106 over wired and/or wireless connection(s).

In scenarios where the analysis platform 106 is implemented partially or entirely on the monitoring device 104, for instance, the measurements 108 may be transferred over a bus from the device’s local storage to a processing system of the device. In scenarios where the monitoring device 104 is configured to generate one or more predictions 110 by processing the measurements 108, the monitoring device 104 may also be configured to provide the generated one or more predictions 110 as output, e.g., by communicating the one or more predictions 110 to an external computing device. In other scenarios, the measurements 108 may be processed by an external computing device configured generate one or more predictions 110. For example, the measurements 108 may be processed by a smartphone associated with the user, a smartphone or other dedicated device associated with the monitoring device 104, and/or one or more server computers at a data center or other location that can be utilized by an entity associated with the monitoring device 104, to name just a few. In other words, those other devices may implement at least a portion of the analysis platform 106 and/or a prediction system 114.

In one or more implementations, the monitoring device 104 is configured to transmit the measurements 108 to an external device over a wired connection with the external device, e.g., via USB-C or some other physical, communicative coupling. Here, a connector may be plugged into the monitoring device 104 or the monitoring device 104 may be inserted into an apparatus having a receptacle that interfaces with corresponding contacts of the device. The measurements 108 may then be obtained from storage of the monitoring device 104 via this wired connection, e.g., transferred over the wired connection to the external device. Such a connection may be used in scenarios where the monitoring device 104 is mailed by the person 102 after the observation period, such as to a health care provider, telemedicine service, provider of the monitoring device 104, or medical testing laboratory.

Alternatively or additionally, the monitoring device 104 may provide the measurements 108 to the analysis platform 106 by communicating the measurements 108 over one or more wireless connections. For example, the monitoring device 104 may wirelessly communicate the measurements 108 to external computing devices, such as a mobile phone, tablet device, laptop, smart watch, other wearable health tracker, and so on. Accordingly, the monitoring device 104 may be configured to communicate with external devices using one or more wireless communication protocols or techniques. By way of example, the monitoring device 104 may communicate with external devices using one or more of Bluetooth (e.g., Bluetooth Low Energy links), near-field communication (NFC), Long Term Evolution (LTE) standards such as 5G, and so forth. Monitoring devices 104 may be configured with corresponding antennae and other wireless transmission means in scenarios where the measurements 108 are communicated to an external device for processing. In those scenarios, the measurements 108 may be communicated to the analysis platform 106 in various manners, such as at predetermined time intervals (e.g., every day, every hour, or every five minutes), responsive to occurrence of some event (e.g., filling a storage buffer of the monitoring device 104), or responsive to an end of an observation period, to name just a few.

Thus, regardless of where the analysis platform 106 is implemented (e.g., at the monitoring device 104, at a smartphone associated with the person 102, or at a server device), the analysis platform 106 obtains the measurements 108 produced by the monitoring device 104. In one or more implementations, the analysis platform 106 also obtains other measurements produced by the monitoring device 104 and/or any other devices used during the observation period, e.g., a smartwatch, chest strap, etc.

In one or more implementations, the analysis platform 106 may be implemented in whole or in part at the monitoring device 104. Alternately or additionally, the analysis platform 106 may be implemented in whole or in part using one or more computing devices external to the monitoring device 104, such as one or more computing devices associated with the person 102 (e.g., a mobile phone, tablet device, laptop, desktop, or smart watch) or one or more computing devices associated with a service provider (e.g., a health care provider, a telemedicine service, a service corresponding to the provider of the monitoring device 104, a medical testing laboratory service, and so forth). In the latter scenario, the analysis platform 106 may be implemented at least in part on one or more server devices.

In the illustrated example 100, the analysis platform includes storage device 112. In accordance with the described techniques, the storage device 112 is configured to maintain the measurements 108 and/or other measurements or information processed by the prediction system 114 to generate one or more predictions 110. The storage device 112 may represent one or more databases and also other types of storage capable of storing the measurements 108 and/or other types of measurements. The storage device 112 may also store a variety of other data, such as personal information, demographic information describing the person 102, information about a health care provider, information about an insurance provider, payment information, prescription information, determined health indicators, account information (e.g., username and password), and so forth. The storage device 112 may also maintain data of other users of a user population.

In the illustrated example 100, the analysis platform 106 also includes the prediction system 114. The prediction system 114 represents functionality to process the measurements 108 to generate the one or more prediction(s) 110. Alternatively or in addition, the prediction system 114 may output one or more time sequences indicating an observation or prediction of one or more events, over time. It is also to be appreciated that the prediction system 114 may output different combinations of multiple predictions in variations.

In at least one implementation, the prediction system 114 uses machine learning to generate one or more predictions 110. By way of example and not limitation, the prediction system 114 may include one or more neural networks trained based on the historical measurements and the historical outcome data of a user population. The prediction system 114 may include one or multiple machine learning models (e.g., an ensemble of models). Alternatively or additionally, the prediction system 114 may include logic (a machine learning model and/or other types of logic) to pre-process the obtained measurements, such as to extract various cardiovascular and/or other features from the sequences of measurements. The illustrated example 100 also includes prediction(s) 110, which corresponds to the output of the prediction system 114.

In various examples, the prediction system 114 is representative of and/or includes a blood pressure monitoring system 116 and the prediction 110 includes and/or is representative of a measurement of blood pressure 118. For instance, as further described in more detail below the blood pressure monitoring system 116 is operable to implement multiparameter cuffless blood pressure monitoring techniques that account for physiological and environmental factors that affect pulse wave characteristics to maintain measurement accuracy over extended periods without recalibration. In various examples, one or more operations of the analysis platform 106, the prediction system 114, and/or the blood pressure monitoring system 116 are performable by one or more of the monitoring device 104, the devices and systems of a healthcare provider, and/or one or more additional devices not shown.

FIG. 2 depicts a nonlimiting example 200 of a monitoring device. The illustrated example 200 depicts the monitoring device 104.

In accordance with the described techniques, the monitoring device 104 includes one or more sensors 202, examples of which include but are not limited to one or more pairs of electrodes, an accelerometer, a pulse oximeter, and sweat sensors, to name just a few. The monitoring device 104 may also include a transmitter 204. In this example 200, the monitoring device 104 further includes one or more adhesive portions 206. In operation, the monitoring device 104 is configured to be applied to the skin via the one or more adhesive portions 206, such that, for example, the one or more sensors 202 are positioned to detect and record the electrical activity of the person 102’s heart, e.g., to produce an electrocardiogram (ECG and/or EKG). In at least one implementation, the monitoring device 104 may be removed by peeling the one or more adhesive portions 206 off of the skin.

It is to be appreciated that the monitoring device 104 and its various components are simply one form factor, and the monitoring device 104 and its components may have different form factors without departing from the spirit or scope of the described techniques.

In one or more implementations, the monitoring device 104 may include a processor and/or memory (not shown). The monitoring device 104, by leveraging the processor, may generate the measurements 108 based on the communications with one or more sensors 202 that are indicative of some aspect of the person 102, such as a blood pressure 118 of the person 102. In one or more implementations, the processor further generates one or more communicable packages of data that include one or more of the measurements 108 and/or other measurements. Alternately or additionally, the processor produces and/or causes storage of other data, which may be used for predicting classifications of physiological conditions, e.g., conditions related to blood pressure 118.

In implementations where the monitoring device 104 is configured for wireless transmission, the transmitter 204 may transmit the measurements 108 wirelessly as a stream of data to a computing device. In one or more implementations, for instance, the monitoring device 104 is configured to transfer (e.g., transmit and/or receive) information (e.g., electrical potential measurements) via a Bluetooth Low Energy (BLE) connection. Alternately or additionally, the monitoring device 104 may buffer the measurements 108 (e.g., in memory) and cause the transmitter 204 to transmit the buffered measurements later at various intervals, e.g., time intervals (every second, every thirty seconds, every minute, every five minutes, every hour, and so on), storage intervals (when the buffered measurements reach a threshold amount of data), and so forth.

### Multiparameter Cuffless Blood Pressure Monitoring System

The following discussion describes techniques that are implementable utilizing the previously and subsequently described systems, components, and devices. Aspects of each of the procedures can be implemented in hardware, firmware, software, or a combination thereof. The procedures are shown as a set of blocks that specify operations that can be performed by one or more devices and are not necessarily limited to the orders shown for performing the operations by the respective blocks. One or more blocks of the procedures, for instance, specify operations that can be programmable by hardware (e.g., processor, microprocessor, controller, firmware) as instructions thereby creating a special purpose machine for carrying out an algorithm as illustrated by the flow diagram. As a result, the instructions are storable on a computer-readable storage medium that causes the hardware to perform the algorithm. In portions of the following discussion, reference will be made to FIGS. 1-13.

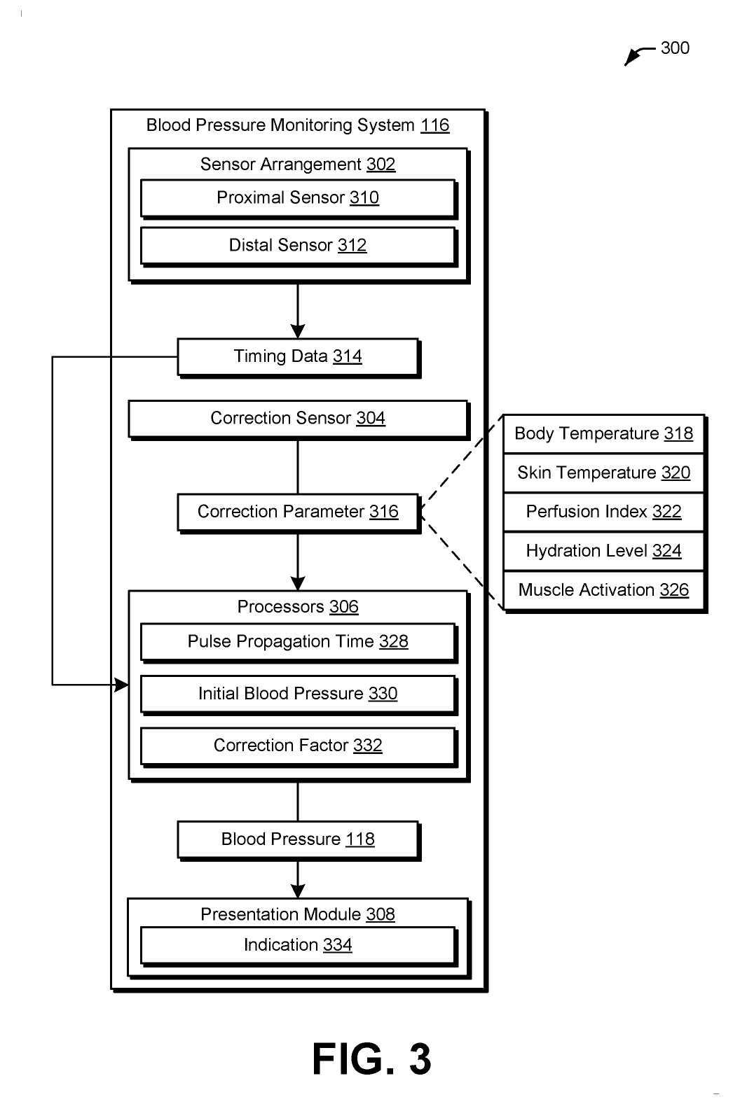

FIG. 3 depicts a nonlimiting system in an example implementation 300 of multiparameter cuffless blood pressure monitoring showing operation of a blood pressure monitoring system 116 in more detail.

In various examples, the blood pressure monitoring system 116 is representative of, supports functionality of, is implementable by, and/or includes (either partially or wholly) a wearable device, such as monitoring device 104, attachable to a skin surface of a user. In additional or alternative examples, the blood pressure monitoring system 116 includes one or more components and/or operations performable external to a wearable device, such as by the analysis platform 106.

The blood pressure monitoring system 116 is operable to generate accurate blood pressure measurements that account for physiological factors that may affect pulse propagation characteristics partially or wholly independent of corresponding blood pressure changes. For instance, various physiological parameters may cause disproportionate effects on pulse propagation time that would otherwise result in inaccurate blood pressure derivations. The blood pressure monitoring system 116 is illustrated to include a sensor arrangement 302, one or more correction sensors 304, and one or more processors 306 that are operable to collect and process physiological data to generate a blood pressure 118, e.g., a derived measurement of blood pressure using the techniques described herein. The blood pressure monitoring system 116 is further illustrated to include a presentation module 308 that is implementable to output the blood pressure 118 and/or perform various functionality using the blood pressure 118.

To begin in this example, the sensor arrangement 302 and correction sensor 304 are configurable to interact with cardiovascular, physiological, and/or environmental systems to collect various measurements for blood pressure determination. For instance, the sensor arrangement 302 is configured to collect physiological timing data 314 that indicates cardiovascular signal characteristics such as electrical activity from the heart, optical changes in blood volume, and/or bioimpedance variations that occur as blood pulses through vessels. The timing data 314 may be processed to derive timing relationships between proximal and distal measurement locations, as further described in more detail below, such as to generate a pulse arrival time (“PAT”) and/or a pulse transit time (“PTT”) along a cardiovascular pathway.

For instance, the sensor arrangement 302 may include a proximal sensor 310 positioned at a proximal location along the cardiovascular pathway and a distal sensor 312 positioned at a distal location along the cardiovascular pathway. The proximal sensor 310 may be configured to detect a first cardiovascular signal as part of the physiological timing data 314, while the distal sensor 312 may be configured to detect a second cardiovascular signal that corresponds to the first cardiovascular signal. In some cases, the proximal sensor 310 includes one or more of a proximal electrocardiogram (“ECG”) sensor, a proximal bioimpedance sensor, or a proximal photoplethysmography (“PPG”) sensor. The distal sensor 312 may include one or more of a distal PPG sensor, a distal pressure sensor, or a distal bioimpedance sensor.

The correction sensor 304 is configured to measure a correction parameter 316 that impacts characteristics (e.g., timing, magnitude, rate, etc.) of pulse propagation independent of a corresponding change to blood pressure. The correction parameter 316 may include various physiological measurements that affect pulse wave characteristics through mechanisms other than blood pressure variations. By way of example, a particular correction parameter 316 may impact blood pressure, however, causes a disproportionate change to the timing data 314 that does not reflect the corresponding blood pressure changes, which can lead to measurement errors if not accounted for. Thus, conventional cuffless blood pressure techniques often experience calibration drift due to presence of one or more correction parameters 316.

A variety of correction parameters 316 are considered. In at least one example, the correction parameter 316 includes body temperature 318 measured using temperature sensors and/or heat flux sensors such as to account for systemic cardiovascular effects on pulse propagation. Additionally or alternatively, the correction parameter 316 may include skin temperature 320 measured adjacent to the sensor arrangement 302 such as to account for an impact of vasomotion (e.g., vasoconstriction and/or vasodilation) on pulse propagation timing. In some examples, the correction parameter 316 includes a perfusion index 322, such as calculated from PPG data collected by the correction sensor 304 such as to account for peripheral circulation impacts on pulse wave characteristics. In various implementations, the correction parameter 316 may include a hydration level 324 measured using bioimpedance analysis such as to account for arterial stiffness variations, and/or muscle activation 326 detected through electromyography to account for arterial wall contraction effects. This is by way of example and not limitation, and various additional or alternative correction parameters 316 are considered.

In various examples, the processors 306 can adjust one or more properties of the sensor arrangement 302 and/or the correction sensor 304 based on the timing data 314 and/or measurements of the correction parameter 316. For instance, the processors 306 may adjust sampling rates of the sensors to optimize signal quality during periods of physiological variability, modify sensor gain settings to accommodate changes in signal amplitude, alter filtering parameters to reduce noise artifacts, selectively activate, or deactivate specific sensors based on detected physiological conditions and so forth. In some examples, the processors 306 may dynamically reconfigure sensor properties, e.g., measurement windows or timing intervals, such as to improve measurement accuracy when correction parameters 316 indicate a likelihood of inaccuracy above a threshold.

The one or more processors 306 are coupled to the sensor arrangement 302 and/or the correction sensor 304 and can generate the blood pressure 118 based on the timing data 314 and/or measurements of the correction parameter 316. The processors 306, for instance, are configured to process the physiological timing data 314 to determine a pulse propagation time 328 based on temporal differences between corresponding points in cardiovascular signals. In various examples, the processors 306 calculate the pulse propagation time 328 as one or more of a pulse transit time (“PTT”) or a pulse arrival time (“PAT”) based on signal analysis from the proximal sensor 310 and distal sensor 312.

By way of example and not limitation, the processors 306 may determine PTT by measuring a time difference between a feature, e.g., an R-wave peak, of an ECG signal detected by the proximal sensor 310 and a corresponding pulse peak in a PPG signal detected by the distal sensor 312. In this example, the PTT represents the time for the pulse wave to travel from a proximal location, e.g., the heart, to a distal location, e.g., a peripheral measurement site. In an alternative or additional example, the processors 306 may determine PAT by measuring a time difference between an ECG feature, e.g., a Q-wave onset, and a corresponding PPG feature, e.g., a “foot” of a corresponding PPG waveform, at the distal location.

Based on the pulse propagation time 328, the processors 306 can generate an initial blood pressure 330, such as by leveraging known relationships between pulse wave velocity/timing and vascular pressure. For instance, the processors 306 may apply empirical models, mathematical algorithms, and/or calibration curves that correlate pulse propagation characteristics to systolic and/or diastolic blood pressure values. In some implementations, the processors 306 may utilize machine learning models trained on population data to establish relationships between pulse propagation time 328 and blood pressure ranges for different demographic groups or physiological conditions.

The processors 306 are then operable to generate the blood pressure 118 based on the initial blood pressure 330 and the correction parameter 316. For instance, the processors 306 can apply a correction factor 332 based on a correction parameter 316 to adjust the initial blood pressure 330 and generate the blood pressure 118. The processors 306 can generate the correction factor 332 based on a variety of considerations, such as but not limited to threshold values of the correction parameter 316, magnitude of deviation of the correction parameter 316 from baseline measurements, rate of change in the correction parameter 316, historical patterns in the correction parameter 316, user-specific calibration data, relationships between one or more correction parameters 316, demographic information, time of day, activity level, posture, environmental conditions, and so forth. The processors 306 can further employ a variety of techniques to generate the correction factor 332, such as but not limited to mathematical algorithms, machine learning models, statistical regression techniques, lookup tables, calibration curves, fuzzy logic systems, neural networks, decision trees, ensemble methods, Bayesian inference, hybrid approaches that combine multiple processing techniques, and so forth.

In at least one example, the processors 306 implement a threshold-based correction where the correction factor 332 is applied responsive to detection that the correction parameter 316 exceeds predetermined ranges. By way of example, the correction parameter 316 includes skin temperature 320. When the skin temperature 320 is above a first threshold, e.g., is above 35°C, the processors 306 may apply a vasodilation correction algorithm that includes a first correction factor 332 to compensate for increased pulse propagation times caused by peripheral vessel expansion. When skin temperature 320 drops below a second threshold, e.g., below 30°C, the processors 306 may activate apply a vasoconstriction correction algorithm that includes a second correction factor 332 to adjust the initial blood pressure 330 to account for shortened pulse propagation times. When the skin temperature 320 is between the first and second thresholds, e.g., between 30°C and 35°C, the processors 306 may apply a reduced and/or no correction factor 332, such as to generate the blood pressure 118 based predominantly on the pulse propagation time 328 such as to conserve computational resources and enable power-efficient operation by selectively activating correction mechanisms when warranted by detected physiological conditions.

In an additional or alternative example, the processors 306 may implement an “always on” correction approach where the correction factor 332 is continuously applied. In such an implementation, the processors 306 may continuously monitor the correction parameter 316 and dynamically adjust the correction factor 332 in real-time based on physiological conditions. For instance, the correction factor 332 may be calculated as a continuous function of the correction parameter 316 where the magnitude of the correction factor 332 varies proportionally with the measured parameter value. This approach may provide nuanced blood pressure adjustments that account for subtle physiological variations.

In one or more examples, the processors 306 may generate one or more correction factors 332 based on multiple correction parameters 316. For instance, the processors 306 may implement a weighting scheme to assign weights to various correction parameters 316 based on a relative impact on pulse propagation time 328, measurement reliability, and/or physiological significance. In an example in which skin temperature 320 and hydration level 324 are measured, the processors 306 may apply a relatively higher weight to skin temperature 320 during periods of significant temperature variation while assigning a lower weight to hydration level 324. The weighting scheme may be dynamically adjusted based on a variety of factors such as signal quality, measurement confidence levels, time-dependent physiological states, user-specific calibration data, relationships between correction parameters 316, and so forth.

In various examples, the processors 306 generate a first correction factor 332 for systolic blood pressure and a second correction factor 332 for diastolic blood pressure. For instance, the first and second correction factors 332 may differ in magnitude and/or direction such as based on how measured correction parameters 316 differentially affect systolic and diastolic measurements. For example, particular physiological conditions (e.g., changes in vascular compliance) may have a relatively larger impact on systolic pressure while other physiological conditions (e.g., changes in peripheral resistance) may cause a relatively larger impact on diastolic pressure.

Accordingly, the blood pressure monitoring system 116 can implement a various techniques to generate the blood pressure 118 based on a variety of considerations. The processors 306 may implement the above described techniques in various temporal scenarios, such as to optimize system performance and accuracy. For instance, the processors 306 may apply correction factors 332 in real-time during data acquisition to provide immediate blood pressure measurements or may perform post-processing analysis of collected timing data 314 and correction parameters 316 to generate retrospective blood pressure calculations with enhanced accuracy. In some implementations, the processors 306 may utilize hybrid approaches that combine real-time preliminary corrections with subsequent post-processing refinements to balance immediate feedback requirements with computational efficiency.

Once generated, the blood pressure monitoring system 116 can perform a variety of functionality based on the blood pressure 118. For instance, the presentation module 308 is configured to output and/or present the blood pressure 118 through various display and communication mechanisms. The presentation module 308 may generate an indication 334 that provides visual, auditory, and/or haptic feedback regarding blood pressure status to the user. In various examples, the presentation module 308 causes display of systolic and diastolic blood pressure values determined by the processors 306, such as based on characteristics of the pulse propagation time 328 and correction parameter 316. The presentation module 308 may also provide information about active correction factors, measurement confidence levels, and trends in blood pressure readings over time. Additionally or alternatively, the presentation module 308 may include wireless transmission capabilities such as to transmit the measurement of the blood pressure 118 to external devices, cloud systems, or healthcare provider networks for further analysis and monitoring. In various embodiments, presentation of the blood pressure 118 includes modification of one or more properties of the wearable device, such as to adjust sensor properties (e.g., sensitivities, collection rates, etc.) of the sensors of the sensor arrangement 302 and/or the correction sensor 304.

In this way, the techniques, devices, and components described herein overcome the limitations of conventional systems through mitigation of inaccuracies due to calibration drift in cuffless blood pressure monitoring and accounting for an impact of various physiological factors on blood pressure measurements. Accordingly, the blood pressure monitoring system 116 can maintain accuracy over extended periods without manual recalibration to support continuous blood pressure monitoring and generation of accurate cardiovascular health insights over an extended wear period. Additionally, selective application of correction factors based on threshold conditions can conserve computational resources by preventing deployment of processing-intensive correction algorithms until warranted by detected physiological parameters. This improves operation of computing devices that implement the described system by reducing unnecessary or redundant calculations during stable physiological conditions, thereby extending battery life and enabling efficient power management in wearable implementations.

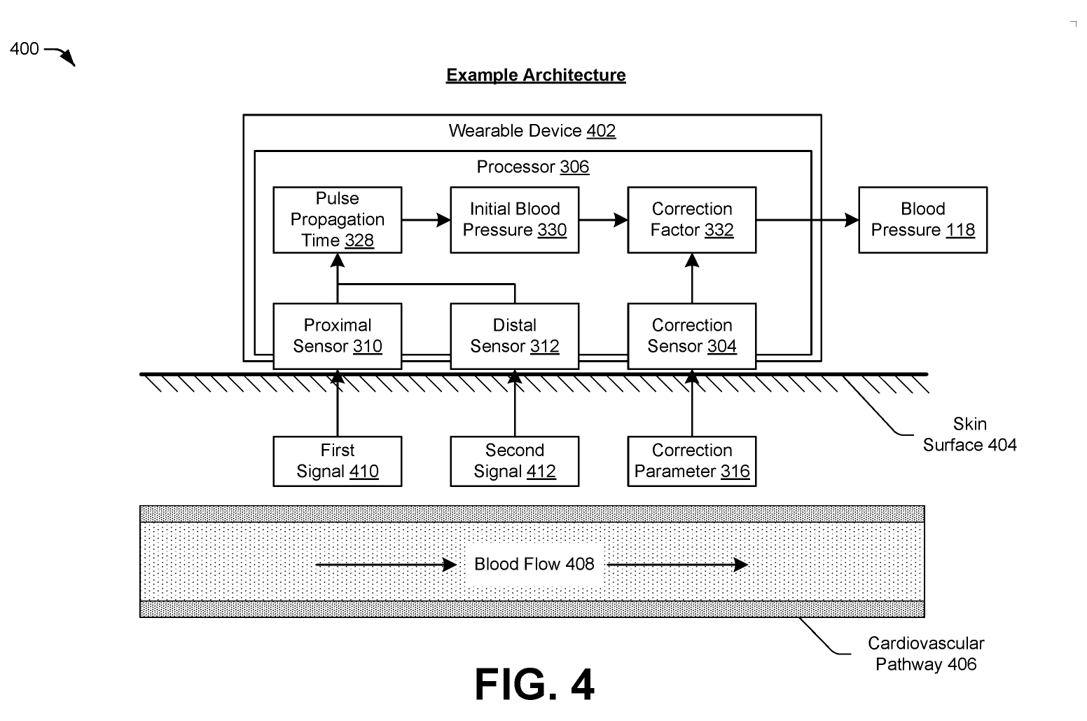

FIG. 4 depicts a nonlimiting example 400 of multiparameter cuffless blood pressure monitoring in which a wearable device generates a blood pressure measurement based on timing data and one or more measured correction parameters.

In this example 400, a wearable device 402 is configured for cuffless blood pressure monitoring through multi-parameter correction, such as through inclusion of one or more components of the blood pressure monitoring system 116. The wearable device 402 may be positioned adjacent to a skin surface 404 of a user, e.g., a person 102, to monitor physiological parameters along a cardiovascular pathway 406 (e.g., an artery, arteriole, capillary, vein, etc.) through which blood flow 408 travels to/from the heart. The wearable device 402 includes a sensor arrangement 302 that incorporates multiple sensors positioned at different locations along the cardiovascular pathway 406 to collect physiological timing data, e.g., the timing data 314, and correction parameters 316 for accurate pulse propagation time measurement.

In the illustrated example, the sensor arrangement 302 includes a proximal sensor 310 positioned at a proximal location along the cardiovascular pathway 406 and a distal sensor 312 positioned at a distal location along the cardiovascular pathway 406. The proximal sensor 310 may be configured to detect a first signal 410 as part of the physiological timing data, while the distal sensor 312 may be configured to detect a second signal 412 as part of the physiological timing data that corresponds to the first signal 410. As described above in more detail, the proximal sensor 310 can include one or more of a proximal ECG sensor, a proximal bioimpedance sensor, or a proximal PPG sensor. The distal sensor 312 may include one or more of a distal PPG sensor, a distal pressure sensor, or a distal bioimpedance sensor. A temporal difference between features of the first signal 410 and the second signal 412 allows for determination of pulse propagation time 328 of a pulse wave along the cardiovascular pathway 406.

The wearable device 402 further includes a correction sensor 304 configured to measure a correction parameter 316 that impacts the pulse propagation time 328 independent of corresponding changes to blood pressure 118. The correction sensor 304 may be integrated within the same form factor as the sensor arrangement 302, such as to allow for simultaneous collection of timing data and correction parameters 316. The processor 306 processes the physiological timing data from the first signal 410 and second signal 412 to determine the pulse propagation time 328, which may include one or more of PTT or PAT.

The processor 306 then generates the blood pressure 118 measurement based on both the pulse propagation time 328 and measurements of the correction parameter 316, such as to compensate for an impact of physiological factors that affect pulse wave velocity independent of blood pressure changes. For instance, the processors 306 generate a correction factor 332 based on characteristics (e.g., a magnitude, frequency, amplitude, duration, rate of change, pattern variability, threshold value, etc.) of the correction parameter 316. The processors 306 then apply the correction factor 332 to the initial blood pressure 330 to generate the blood pressure 118.

The wearable device 402 can output the blood pressure 118 in various ways, such as through display on an integrated screen, transmission to a paired mobile device, storage in local memory for later retrieval, wireless communication to cloud-based health platforms, audible alerts or notifications, haptic feedback patterns, visual indicators such as LED lights, integration with electronic health record systems, real-time streaming to healthcare monitoring services, or combinations thereof. The presentation module 308 may select appropriate output methods based on user preferences, device capabilities, connectivity status, urgency of readings, or clinical requirements.

While in this example 400 the proximal sensor 310, distal sensor 312, and correction sensor 304 are illustrated as included within a housing of the wearable device 402, this is by way of example and not limitation. In one or more examples, one or more of the processor 306, proximal sensor 310, distal sensor 312, and/or correction sensor 304 are positioned remote from the wearable device 402, such as included in a separate monitoring device. For instance, one or more of the sensors and/or components of the blood pressure monitoring system 116 may be distributed across multiple wearable devices or form factors to provide comprehensive physiological monitoring. For example, the blood pressure monitoring system 116 may be implemented across a combination of devices such as a chest patch for proximal measurements, a wrist-worn device for distal measurements, and a ring-based sensor for additional correction parameters. Thus, the techniques described herein are able to incorporate data collected from various devices at various physiological locations to provide a variety of insights.

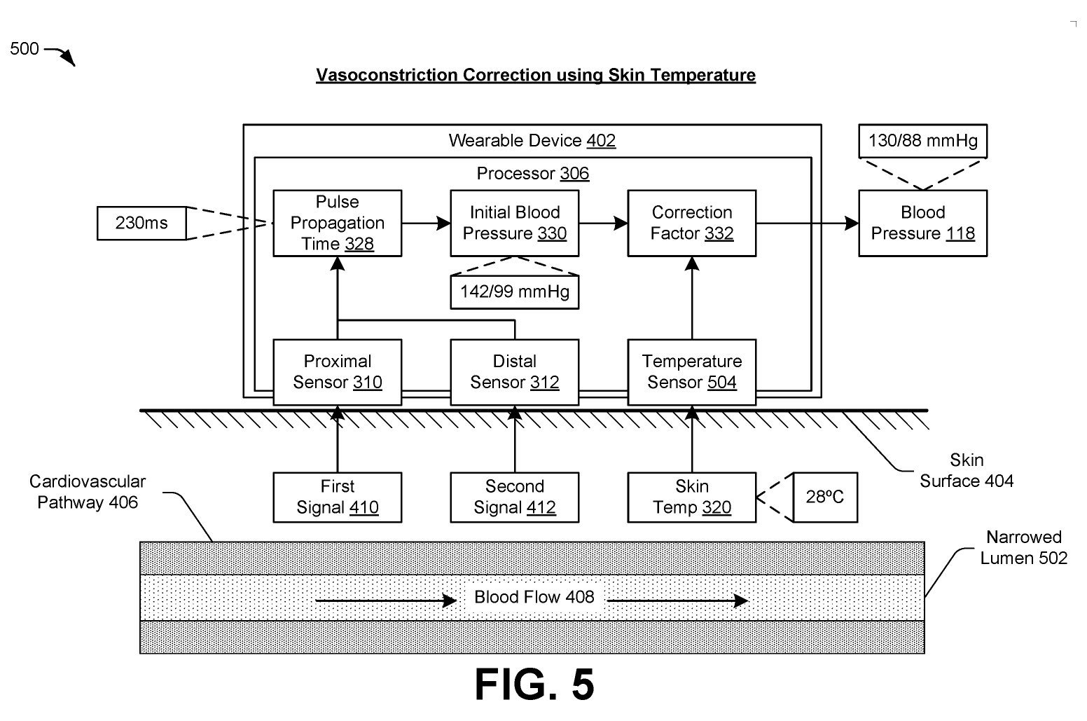

FIG. 5 depicts a nonlimiting example 500 of multiparameter cuffless blood pressure monitoring in which a temperature sensor is used to collect skin temperature data as a correction parameter.

In this example 500, the cardiovascular pathway 406 is subject to vasoconstriction, such as a result of cold exposure. The cold temperature, for instance, causes arterial walls to contract and reduces an internal diameter of blood vessels, as illustrated by a narrowed lumen 502 of the cardiovascular pathway 406. The narrowed lumen 502 affects characteristics of blood flow 408 and resulting pulse propagation measurements, which would otherwise compromise accuracy of blood pressure. For instance, the reduced diameter of the narrowed lumen 502 may cause pulse waves to travel relatively faster through the constricted cardiovascular pathway 406, without a proportional change to blood pressure.

Thus, a pulse propagation time 328 based on a temporal difference between features of a first signal 410 and a second signal 412 is relatively short, e.g., 230ms relative to a baseline time of 250ms. Based on the pulse propagation time 328, the processor 306 generates a measurement of an initial blood pressure 330 of 142/99 mmHg. Thus, the initial blood pressure 330 based solely on the pulse propagation time 328 and without correction for vasoconstriction is artificially elevated relative to an actual blood pressure of the person 102.

Accordingly, the correction sensor 304 in this example includes a temperature sensor 504, which is operable to measure skin temperature 320 adjacent to the sensor arrangement 302 as a correction parameter 316 such as to account for an impact of vasomotion on the pulse propagation time 328. In the example 500, the temperature sensor 504 detects a skin temperature 320 of 28 degrees Celsius, e.g., four degrees below a baseline of the particular user. The processor 306 generates a correction factor 332 based on the skin temperature 320 measurements.

The processor 306 then applies the correction factor 332 based on the measured skin temperature 320 to generate an adjusted blood pressure 118 that accounts for the vasomotion effects. In the example 500, the correction factor 332 adjusts the initial blood pressure 330 from 142/99 mmHg to a corrected blood pressure 118 of 130/88 mmHg. This correction compensates for the faster pulse propagation caused by the narrowed lumen 502 and provides an accurate representation of blood pressure that is independent of peripheral vascular changes to pulse propagation time 328.

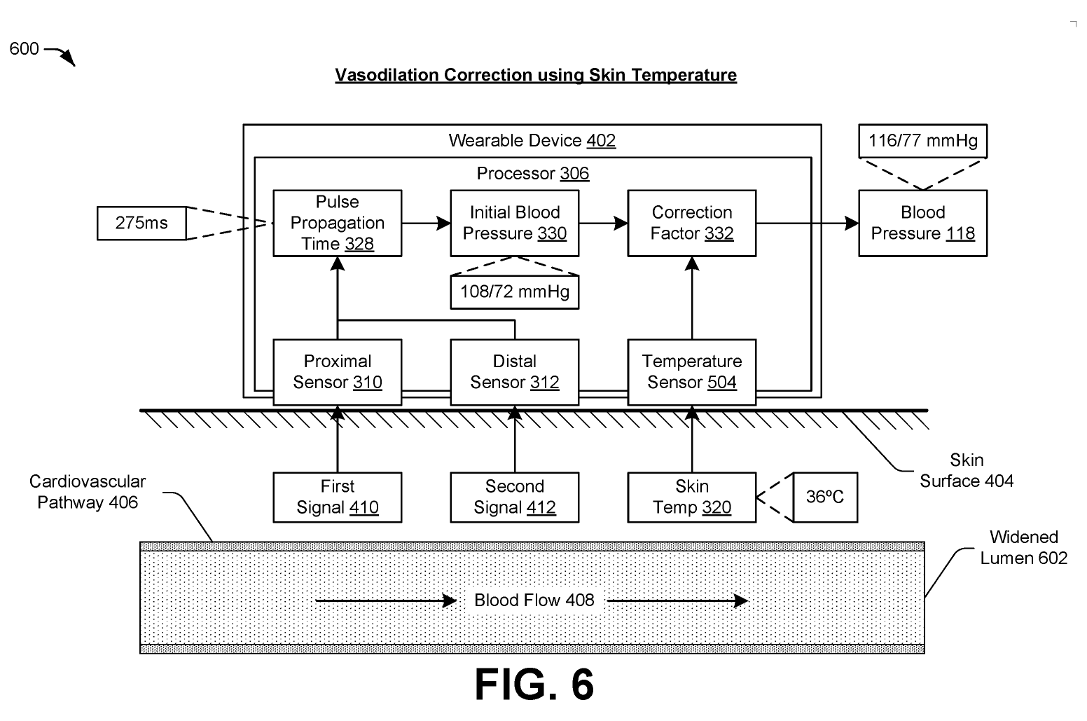

FIG. 6 depicts an additional nonlimiting example 600 of multiparameter cuffless blood pressure monitoring in which a temperature sensor is used to collect skin temperature data as a correction parameter.

In this example 600, the cardiovascular pathway 406 is subject to vasodilation, such as a result of heat exposure. The elevated temperature causes arterial walls to relax and increases the internal diameter of blood vessels, as illustrated by a widened lumen 602 of the cardiovascular pathway 406. The widened lumen 602 affects characteristics of blood flow 408 and resulting pulse propagation measurements, such as to cause pulse waves to travel relatively slower through the dilated cardiovascular pathway 406.

Accordingly, a pulse propagation time 328 based on a temporal difference between a first signal 410 and a second signal 412 is relatively long, e.g., 275ms relative to a baseline time of 250ms. Based on the pulse propagation time 328, the processor 306 generates a measurement of an initial blood pressure 330 of 108/72 mmHg. Accordingly, the initial blood pressure 330 based solely on the pulse propagation time 328 and without correction for vasodilation is inaccurately low relative to an actual blood pressure of the user.

To account for this, the temperature sensor 504 in the illustrated example detects a skin temperature 320 of 36 degrees Celsius, e.g., four degrees above a baseline temperature for the user. The processor 306 generates a correction factor 332 based on the skin temperature 320 and applies the correction factor 332 to the initial blood pressure 330 to account for the vasomotion effects. In the example, the correction factor 332 adjusts the initial blood pressure 330 from 108/72 mmHg to a corrected blood pressure 118 of 116/77 mmHg. This correction compensates for the relatively slow pulse propagation time 328 caused by the widened lumen 602 and thus provides an accurate representation of blood pressure 118.

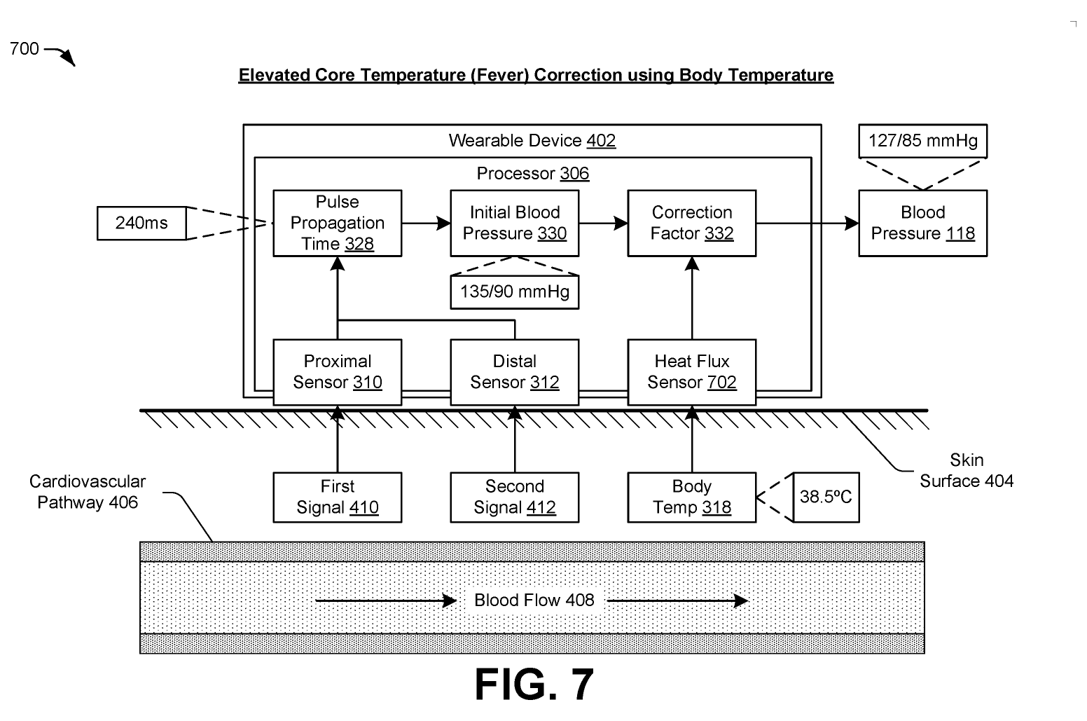

FIG. 7 depicts a nonlimiting example 700 of multiparameter cuffless blood pressure monitoring in which a heat flux sensor is used to collect body temperature data as a correction parameter.

In this example, the wearable device 402 incorporates a heat flux sensor 702 as a correction sensor 304 to measure body temperature 318 as a correction parameter 316, such as to account for systemic cardiovascular effects on pulse propagation time 328. The heat flux sensor 702 is operable to detect thermal energy transfer between the body of the user and a surrounding environment and thus can provide measurements of core body temperature variations (such as due to fever, dehydration, activity, physical exertion, metabolic changes, etc.) that may affect cardiovascular function. In some cases, the heat flux sensor 702 may be positioned adjacent to the skin surface 404 to optimize thermal contact and measurement accuracy. In this example, the body temperature 318 is elevated, e.g., 38.5 degrees C, such as due to a fever.

When body temperature 318 increases above normal physiological ranges, systemic changes occur in cardiovascular function that may alter a relationship between pulse propagation time 328 and actual blood pressure. For instance, a pulse propagation time 328 is relatively quick, e.g., 240ms, and thus an initial blood pressure 330 generated based solely on the pulse propagation time 328 is inaccurately high, e.g., 135/90 mmHg.

To account for the elevated body temperature 318, the processors 306 generate a correction factor 332 based on the measurements from the heat flux sensor 702 and apply the correction factor 332 to the initial blood pressure 330. In the example, the processor 306 applies the correction factor 332 to adjust the initial blood pressure 330 from 135/90 mmHg to a corrected blood pressure 118 of 127/85 mmHg, which provides an accurate representation of blood pressure 118.

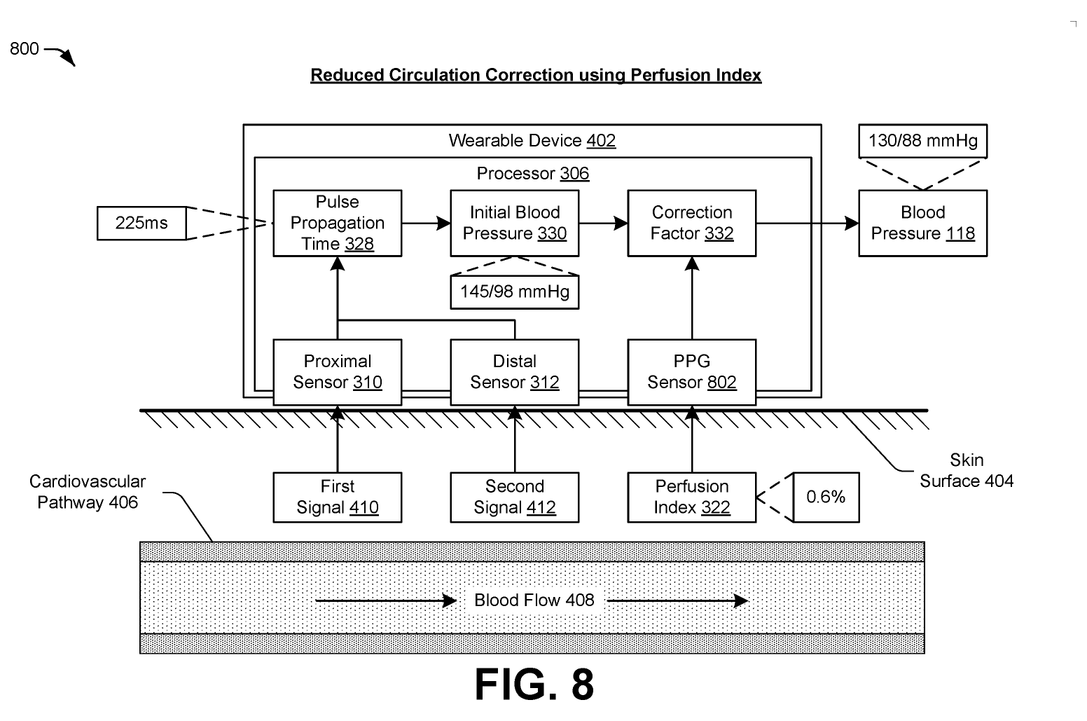

FIG. 8 depicts a nonlimiting example 800 of multiparameter cuffless blood pressure monitoring in which a PPG sensor is used to collect perfusion index data as a correction parameter.

In this example 800, the wearable device 402 incorporates a PPG sensor 802 to measure perfusion index 322 as a correction parameter 316, such as to account for an impact of peripheral circulation characteristics on pulse propagation time 328. The PPG sensor 802 is operable to collect various physiological data for the processor 306 to calculate a ratio of pulsatile to non-pulsatile blood flow, which provides an indication of peripheral circulation. In various examples, changes to peripheral circulation cause changes in cardiovascular function that may alter a relationship between pulse propagation time 328 and actual blood pressure.

For instance, in this example the perfusion index 322 is relatively low, e.g., 0.6%, which results in a relatively quick pulse propagation time 328, e.g., 225ms, and thus an initial blood pressure 330 generated based solely on the pulse propagation time 328 is inaccurately high, e.g., 145/98 mmHg. To account for the relatively low perfusion index 322, the processor 306 generates a correction factor 332 based on the measurements from the PPG sensor 802 and applies the correction factor 332 to the initial blood pressure 330. In the illustrated example, the correction factor 332 adjusts the initial blood pressure 330 from 145/98 mmHg to a corrected blood pressure 118 of 130/88 mmHg, which is biologically accurate.

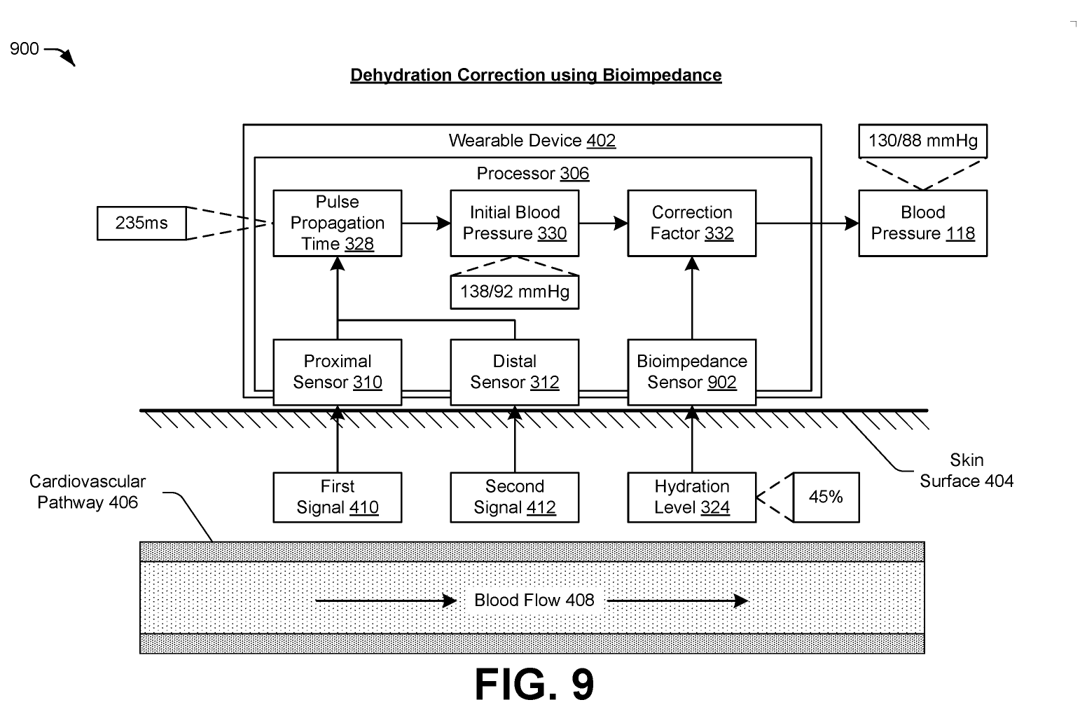

FIG. 9 depicts a nonlimiting example 900 of multiparameter cuffless blood pressure monitoring in which a bioimpedance sensor is used to collect hydration level data as a correction parameter.

In this example, the wearable device 402 incorporates a bioimpedance sensor 902 that measures hydration level 324 as a correction parameter 316, such as to account for an impact of arterial stiffness due to tissue dehydration on the pulse propagation time 328. For instance, the bioimpedance sensor 902 stimulates electrodes to collect impedance data for hydration estimation, such as to provide a quantitative assessment of a fluid status of various tissues within the body. In an example, the bioimpedance sensor 902 applies an electrical current through electrodes positioned on the skin surface 404 and measures resulting impedance characteristics of body tissues. Body tissues with relatively high water content, for instance, exhibit relatively low electrical impedance, while dehydrated tissues present relatively high impedance values.

The bioimpedance sensor 902 analyzes these impedance variations to determine the hydration level 324, which in various examples correlates to a fluid content of blood vessels and/or surrounding tissues. In this example, the hydration level 324 is determined to be 45%, which is decreased from a baseline hydration level of 55%. The reduced hydration level may cause arterial walls to become stiff and less compliant, which can affect pulse wave velocity and alter the pulse propagation time 328 independent of actual blood pressure changes.

For instance, the pulse propagation time 328 in this example is measured to be relatively quick, e.g., 235ms, and the processor 306 calculates an initial blood pressure 330 of 138/92 mmHg based on this pulse propagation time 328 measurement. However, dehydration conditions may cause arterial walls to become stiffer, which affects the relationship between pulse propagation time 328 and actual blood pressure 118, and the initial blood pressure 330 is inaccurately elevated from an actual blood pressure. Accordingly, the processor 306 generates a correction factor 332 based on the hydration level 324, and applies the correction factor 332 to the initial blood pressure 330 to generate a measurement of blood pressure 118, e.g., 130/88 mmHg, that accounts for the impact of dehydration on pulse wave velocity and provides an accurate representation of actual cardiovascular pressure conditions.

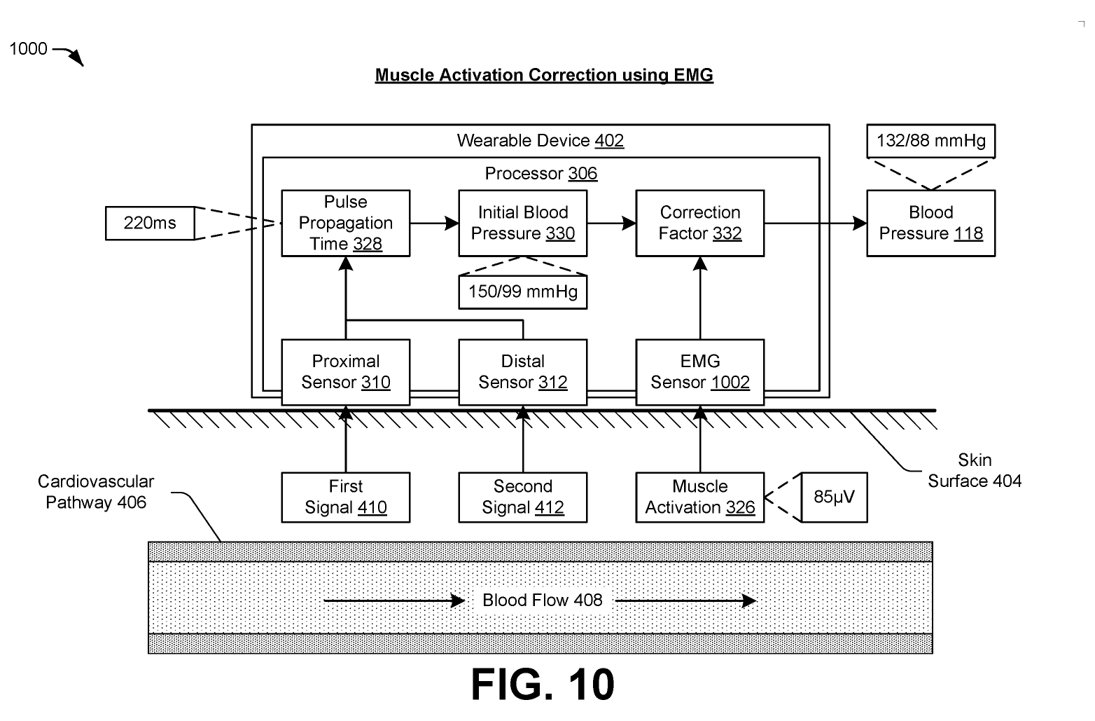

FIG. 10 depicts a nonlimiting example 1000 of multiparameter cuffless blood pressure monitoring in which an electromyography (“EMG”) sensor is used to collect muscle activation data as a correction parameter.

In this example, the wearable device 402 includes an EMG sensor 1002 as a correction sensor 304 to measure muscle activation 326 as a correction parameter 316, such as to account for arterial wall contractility on the pulse propagation time 328. For instance, muscle activation 326 may influence pulse propagation time 328 through changes in arterial wall tension and compliance that occur independently of blood pressure 118 variations. The EMG sensor 1002 may detect electrical activity generated by smooth muscle contractions within arterial walls, such as to provide measurements of muscular influences on vascular characteristics that impact pulse propagation time 328 measurements.

For example, as illustrated the muscle activation 326 is measured to be relatively elevated, e.g., 85µV. Accordingly, the pulse propagation time 328 is relatively fast, e.g., 220ms, which causes the initial blood pressure 330 generated by the processor 306 based on the pulse propagation time 328 to be inaccurately high, e.g., 150/99 mmHg. Accordingly, the processor 306 generates a correction factor 332 based on the data collected by the EMG sensor 1002 and applies the correction factor 332 to the initial blood pressure 330 to generate a measurement of blood pressure 118, e.g., 132/88 mmHg, that considers the impact of muscle activation 326.

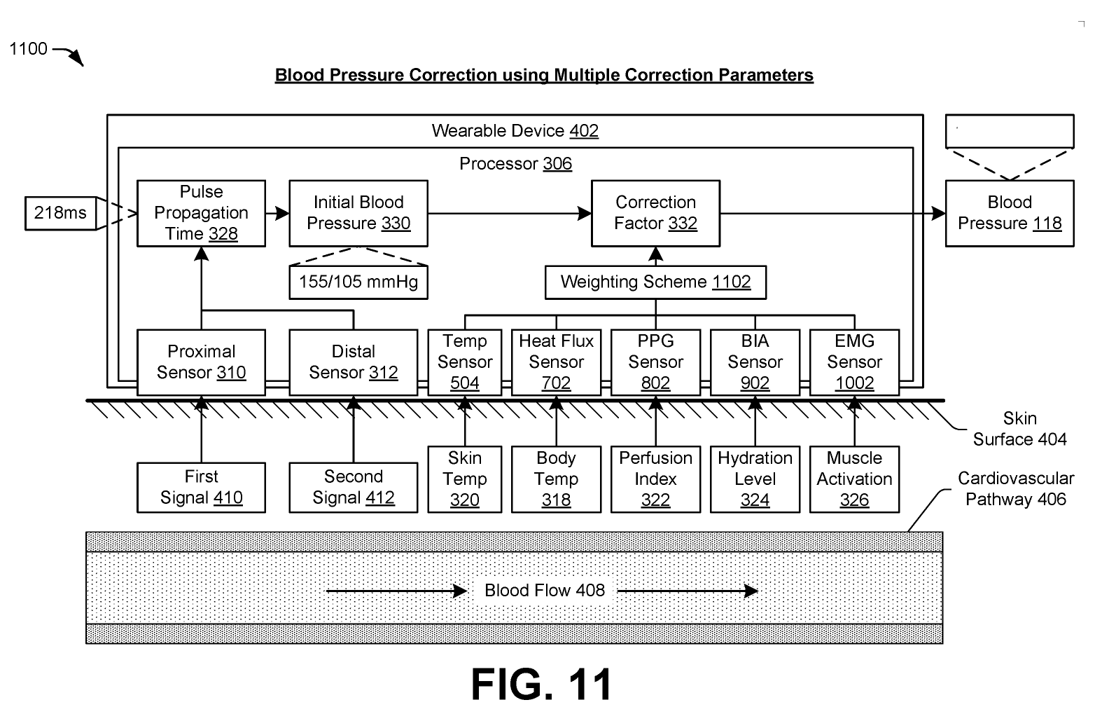

FIG. 11 depicts a nonlimiting example 1100 of multiparameter cuffless blood pressure monitoring in which multiple correction parameters are used to generate a blood pressure measurement.

In this example 1100, the wearable device 402 includes a variety of correction sensors 304, such as a temperature sensor 504, a heat flux sensor 702, a PPG sensor 802, a bioimpedance sensor 902 (“BIA”), and an EMG sensor 1002. The processor 306 may coordinate measurements from various correction sensors to generate a correction factor 332 based on various correction parameters 316 (e.g., skin temperature 320, body temperature 318, perfusion index 322, hydration level 324, and/or muscle activation 326) for application to the initial blood pressure 330.

In various examples, the processor 306 may implement a weighting scheme 1102 to weight contributions of one or more of the correction parameters 316 in generating the correction factor 332. The weighting scheme 1102, for instance, applies weights to each correction parameter 316 based on factors such as measurement reliability, physiological relevance, magnitude, rates of change, temporal stability, and so forth. In some implementations, the weighting scheme 1102 assigns relatively high weights to correction parameters that demonstrate stronger correlation with pulse propagation time 238 variations independent of blood pressure changes.

In some examples, the processor 306 adjusts the weights based on a relationship between two or more of the correction parameters 316. For instance, when both skin temperature 320 and perfusion index 322 indicate peripheral vasoconstriction, the processor 306 may increase the weight assigned to these parameters relative to an example in which the parameters indicate contradictory physiological states. Similarly, if hydration level 324 decreases while body temperature 318 increases, the processor 306 may increase a weight of the hydration measurement, e.g., due to amplification of temperature effects on vascular compliance by dehydration. The processor 306 is operable to dynamically adjust the weights within the weighting scheme 1102 based on real-time assessment of features of the correction parameters 316, such as magnitude, rate of change, signal quality, measurement confidence levels from each correction sensor 304, and so forth.

In some examples, the correction parameter measurements may be incorporated through continuous integration into blood pressure calculation algorithms executed by the processor 306. In such examples, the processor 306 continuously monitors the correction parameters 316 and applies real-time adjustments to the blood pressure calculation. This continuous integration allows the processor 306 to account for gradual physiological changes that may affect the pulse propagation time 328 over extended monitoring periods.

Alternatively or additionally, the processor 306 may apply the correction factor 332 to the initial blood pressure 330 responsive to detection that one or more of the correction parameters 316 exceed a threshold. For instance, the processor 306 monitors each correction parameter 316 against predetermined threshold values that indicate when physiological conditions may significantly impact pulse propagation time. When the processor 306 detects that one or more correction parameters 316 exceed the established thresholds, the processor 306 activates one or more correction algorithms and/or increases a weighting of affected parameters within the weighting scheme 1102. The threshold-based approach allows the processor 306 to conserve computational resources during periods of stable physiological conditions while providing enhanced correction when appropriate.

The processor 306 may implement various computational approaches to generate the blood pressure 118. For instance, the processor 306 may utilize one or more mathematical algorithms that apply predetermined correction equations to adjust the initial blood pressure 330 such as based on the weighted combination of correction parameters 316. As further described in more detail below, in some examples the processor 306 employs one or more machine learning models trained to calibrate blood pressure readings based on correction parameters 316. For instance, a particular machine learning model is trained to understand complex relationships between multiple correction parameters 316 and respective effects on pulse propagation time 328.

The processor 306 may also implement statistical regression techniques that establish mathematical relationships between correction parameters 316 and blood pressure adjustments through analysis of training data. Additionally or alternatively, the processor 306 may access lookup tables that provide predetermined correction factors 332 for various combinations of correction parameter ranges or utilize calibration curves that map correction parameter values to corresponding blood pressure adjustments.

The processor 306 may determine a systolic blood pressure value and/or a diastolic blood pressure value to include as part of the blood pressure 118 such as based on characteristics of the pulse propagation time 328 and/or the measurements of the correction parameters 316. In one or more examples, the correction parameters 316 may differentially affect systolic and diastolic measurements, and thus the processor 306 is operable to apply separate correction factors 332 and/or weighting schemes 1102 for each blood pressure component.

In the illustrated example, the processors 306 generates the pulse propagation time 328 based on the first signal 410 and second signal 412. The pulse propagation time 328 is relatively quick, e.g., 218ms, and thus the initial blood pressure 330 is inaccurately elevated, e.g., 155/105mmHg. Accordingly, the processor 306 applies the correction factor 332 based on of the various correction parameters 316 to generate a physiologically accurate measurement of blood pressure 118 of 139/95mmHg. Accordingly, the techniques described herein can leverage multiple physiological measurements to maintain measurement accuracy across diverse physiological conditions and individual variations.

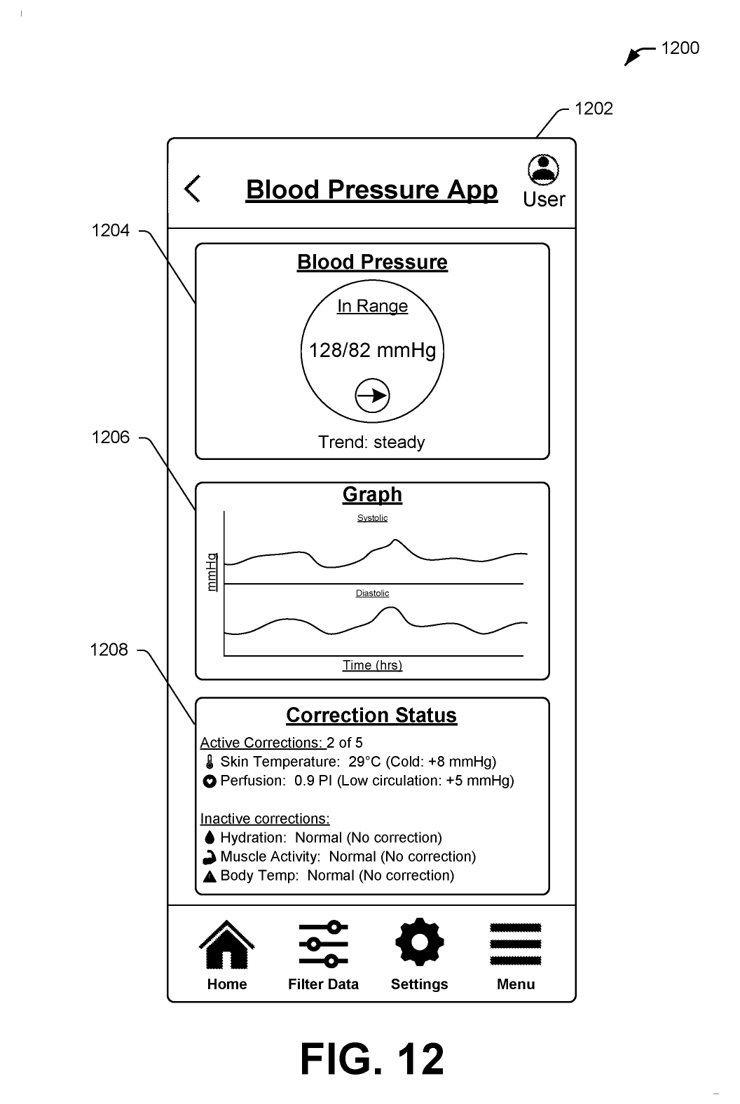

FIG. 12 depicts a nonlimiting example 1200 of multiparameter cuffless blood pressure monitoring in which a user interface 1202 for a blood pressure monitoring scenario is shown.

The user interface 1202 enables users to monitor blood pressure trends and view a status of various correction parameters that may impact measurement accuracy. For instance, the user interface 1202 includes a first panel 1204 that includes blood pressure readings and status information. In this example, the first panel 1204 displays a blood pressure measurement of “128/82 mmHg” along with a status indicator showing “In Range”, such as to communicate that the measured values fall within normal parameters. Additionally, the first panel 1204 includes a trend indicator that shows “steady” to inform users about a recent direction of blood pressure changes. In some implementations, the first panel 1204 may include color coding or other visual indicators to communicate whether blood pressure readings are within target ranges or require attention.

Below the first panel 1204, a second panel 1206 includes a graphical representation of blood pressure trends over time. The second panel 1206 presents systolic and diastolic blood pressure measurements as separate trend lines that provides a visual representation of patterns and variations in blood pressure over an extended period. The graph included in the second panel 1206 can depict trends, fluctuations, and/or periods of stability in blood pressure measurements. In various implementations, the second panel 1206 may include interactive features that allow users to zoom in on particular time periods and/or view detailed measurement data for particular points along the trend lines.

A third panel 1208 is positioned beneath the second panel 1206 and displays correction status information related to the various correction parameter 316 that may impact pulse propagation time measurements. The third panel 1208 provides status indicators for multiple correction parameters 316 including temperature, perfusion, hydration, muscle activity, and body temperature measurements. Each correction parameter in the third panel 1208 may be displayed with an active or inactive status, such as to indicate whether corrections are currently being applied based on the measured values of the respective correction parameters 316. For example, the third panel 1208 in this examples shows that corrections based on skin temperature and perfusion are currently active, while corrections based on hydration, muscle activity, and body temperature remain inactive. This is by way of example and not limitation, and the user interface 1202 can be configured in a variety of ways and include various features.

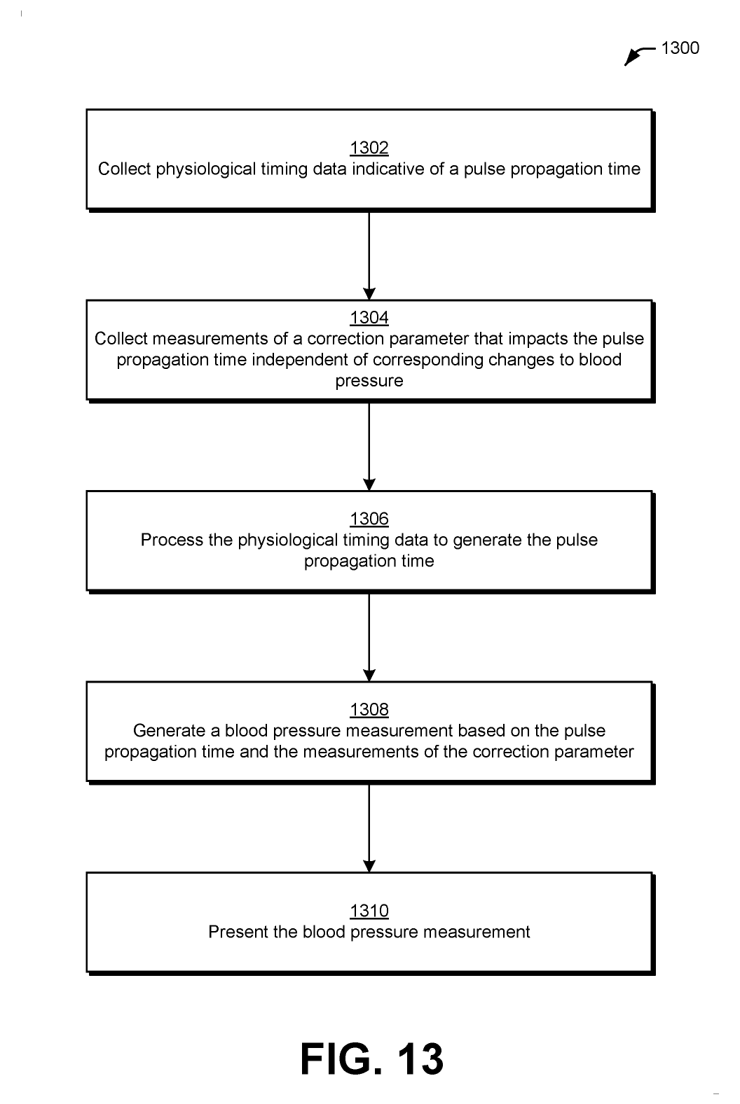

FIG. 13 depicts a flow diagram depicting an algorithm as a step-by-step procedure 1300 in an example implementation, one or more steps of which are performable by a processing device to generate a blood pressure 118 using multiparameter cuffless blood pressure monitoring techniques.

To begin in this example, physiological timing data indicative of a pulse propagation time is collected (block 1302). The timing data 314, for instance, may be collected by one or more sensors of a sensor arrangement 302 of a wearable device 402 during a monitoring period. For instance, the timing data 314 includes a first physiological signal collected by a proximal sensor 310 disposed at a proximal location along a cardiovascular pathway 406 and a second physiological signal collected by a distal sensor 312 disposed at a distal position along the cardiovascular pathway 406. A variety of types of data can be included in the first signal 410 and/or the second signal 412, such as but not limited to various combinations of ECG data, PPG data, bioimpedance data, and so forth. Accordingly, the timing data 314 can indicate temporal differences between corresponding points in the first physiological signal and the second physiological signal.

Measurements of a correction parameter that impacts the pulse propagation time independent of corresponding changes to blood pressure are collected (block 1304). For instance, the wearable device 402 further includes one or more correction sensors 304 to collect measurements of one or more correction parameters 316. The correction parameter may include at least one of body temperature 318, skin temperature 320, perfusion index 322, hydration level 324, or muscle activation 326. The correction sensor 304 measures these parameters to account for physiological factors that affect pulse propagation measurements, e.g., pulse propagation time 328, partially or wholly independent from actual blood pressure changes.

The physiological timing data is processed to generate the pulse propagation time (block 1306). For instance, processors 306 are operable to analyze the first physiological signal and the second physiological signal to determine a pulse propagation time 328, such as a pulse transit time (“PTT”) and/or a pulse arrival time (“PAT”) based on temporal differences between corresponding points in the physiological timing data 314. For example, processors 306 may identify characteristic features in the first physiological signal, such as an R-wave peak in an ECG signal, and corresponding features in the second physiological signal, such as a pulse arrival at the distal location as indicated by PPG data. The processors can determine a temporal difference between these corresponding features to generate the pulse propagation time 328.

A blood pressure measurement is then generated based on the pulse propagation time and the measurements of the correction parameter (block 1308). For example, the processors 306 may generate an initial blood pressure 330 based on the pulse propagation time 328 and adjust the initial blood pressure 330 based on the measurements of the correction parameter 316. For instance, the processors 306 may apply a correction factor 332 to the initial blood pressure 330 based on measurements of the correction parameter 316.

In some examples, adjustment of the initial blood pressure 330 by the correction factor 332 is performed responsive to detection that one or more of the correction parameters 316 are above or below a threshold. As such, the processors 306 may implement different processing modalities above and below thresholds. The processors 306 may further perform real-time optimization of device operation by switching between different processing modes based on threshold conditions, such as activating temperature-based corrections when body temperature 318 deviates from baseline ranges or implementing hydration-based adjustments when hydration level 324 falls below predetermined values.

The blood pressure measurement is then presented (step 1310). For instance, the blood pressure monitoring system 116 can perform various functionality based on the generated measurement of the blood pressure 118. In one or more examples, the blood pressure monitoring system 116 can output an indication 334 of the blood pressure 118 via various display methods or communication interfaces. The indication 334 may include systolic and diastolic values that account for the measured correction parameters 316. The indication 334 may include visual indicators, numerical displays, or trend information that communicates the blood pressure status to users or healthcare providers.

In some examples, the blood pressure 118 may be transmitted (e.g., wirelessly) to external devices and/or stored locally for subsequent analysis. In at least one example, the processors 306 are operable to change one or more conditions of the wearable device 402 based on the blood pressure 118, such as to increase sampling rates, activate/deactivate one or more sensors, and so forth. Accordingly, the techniques described herein enable continuous and/or periodic blood pressure monitoring with enhanced accuracy through the systematic incorporation of correction parameters that address common sources of measurement drift in cuffless blood pressure devices.

### Machine Learning in Multiparameter Cuffless Blood Pressure Monitoring Systems

The previous examples describe various instances of machine-learning models such as with respect to the blood pressure monitoring system 116 and/or the prediction system 114. In one or more examples, a machine-learning model (e.g., an AI model) refers to a computer representation that is tunable (e.g., through training and retraining) based on inputs without being actively programmed by a user to approximate unknown functions, automatically and without user intervention. For instance, the term machine-learning model includes a model that utilizes algorithms to learn from, and make predictions on, known data by analyzing training data to learn and relearn to generate outputs that reflect patterns and attributes of the training data.

In the context of multiparameter cuffless blood pressure monitoring, machine-learning models are implementable (e.g., by one or more processors 306 of the blood pressure monitoring system 116) to analyze physiological data patterns and correction parameters 316 to generate accurate measurements of blood pressure 118 that account for calibration drift. For example, the processors 306 may utilize one or more machine-learning models to process physiological timing data 314 (such as ECG readings, PPG waveforms, bioimpedance measurements, etc.) collected by the sensor arrangement 302 and/or correction parameters 316 including body temperature 318, skin temperature 320, perfusion index 322, hydration level 324, and muscle activation 326 collected by the correction sensors 304.

Examples of machine-learning models applicable to multiparameter blood pressure monitoring include neural networks, convolutional neural networks (“CNNs’”) such as for analyzing pulse waveform characteristics, long short-term memory (“LSTM”) neural networks such as to analyze temporal patterns in pulse propagation time 328 and correction parameters 316, generative adversarial networks (“GAN’s”), decision trees (e.g., for threshold-based correction activation), support vector machines (“SVM’s”), linear regression models for establishing relationships between correction parameters 316 and blood pressure adjustments, logistic regression for binary correction factor activation, Bayesian networks for probabilistic correction factor determination, random forest learning for feature importance in various weighting schemes 1102, dimensionality reduction algorithms, boosting algorithms, deep learning neural networks, and so forth.

A machine-learning model, for instance, is configurable using a plurality of layers having, respectively, a plurality of nodes. The plurality of layers are configurable to include an input layer, an output layer, and one or more hidden layers. In the context of multiparameter cuffless blood pressure monitoring, the input layer may receive various physiological parameters from the measurements 108, such as pulse propagation time 328 features, correction parameters 316 including temperature measurements, perfusion index 322 values, hydration level 324 data, muscle activation 326 signals, and so forth. The hidden layers, for instance, process these inputs through weighted connections to identify complex relationships between pulse propagation characteristics and correction parameters 316 that affect blood pressure accuracy, e.g., relationships that are not detectable using conventional calibration modalities. The output layer may produce a measurement of blood pressure 118 that incorporates correction factors 332 based on the measured correction parameters 316 or generate weighting schemes 1102 for combining multiple correction parameters 316. Calculations are performed by the nodes within the layers via hidden states through a system of weighted connections that are “learned” during training of the machine-learning model to implement a variety of blood pressure calibration and correction tasks.

In order to train the machine-learning model for multiparameter blood pressure monitoring, training data is received that provides examples of “what is to be learned” by the machine-learning model, i.e., as a basis to learn patterns from the data. For multiparameter blood pressure monitoring applications, the training data may include labeled datasets of physiological measurements from individuals with known blood pressure values measured using reference cuff-based devices, along with corresponding pulse propagation times 328 and correction parameters 316 such as temperature variations, hydration levels, perfusion indices, and muscle activation states. A machine-learning system that includes the machine learning model, for instance, collects and preprocesses the training data that includes input features (e.g., PTT measurements, PAT measurements, skin temperature 320 readings, body temperature 318 values, perfusion index 322 calculations, hydration level 324 assessments, muscle activation 326 signals) and corresponding target labels, e.g., accurate systolic and diastolic blood pressure values, appropriate correction factors 332, optimized weighting schemes 1102 for multiple correction parameters 316, etc.

The machine-learning system is further operable to initialize various parameters of the machine-learning model, which are usable by the machine-learning model as internal variables to represent and process information during training. These parameters are further usable to represent interferences gained through training regarding relationships between pulse propagation characteristics and physiological factors that cause calibration drift. In one or more implementations, the training data is separated into batches to improve processing and optimization efficiency of the parameters of the machine-learning model during training, which is particularly beneficial for model accuracy when processing large volumes of physiological time-series data including pulse timing measurements and correction parameter variations over extended monitoring periods.

The training data is then received as an input by the machine-learning model and used as a basis for generating predictions based on a current state of parameters of layers and corresponding nodes of the model, a result of which is output as output data, e.g., a corrected blood pressure 118, correction factor 332 values, weighting scheme 1102 parameters, etc. For example, the blood pressure monitoring system 116 includes a machine-learning model that is trained to recognize patterns in pulse propagation time 328 variations and correction parameters 316 that correlate with accurate blood pressure measurements, which enables the processors 306 to generate precise blood pressure 118 values that account for physiological factors affecting pulse wave characteristics independent of actual blood pressure changes.

Training of the machine-learning model can include calculation of a loss function to quantify a loss associated with operations performed by nodes of the machine learning model. The loss function is configurable in various ways to control operation and/or functionality of the machine learning model. For instance, the loss function may be designed to prioritize accuracy in blood pressure estimation while minimizing calibration drift over extended monitoring periods and accounting for individual physiological variations in correction parameter responses. Calculation of the loss function, for instance, includes comparing a difference between predictions specified in the output data (e.g., predicted blood pressure values with applied correction factors 332) with target labels specified by the training data (e.g., reference cuff-based blood pressure measurements). The loss function is configurable in a variety of ways, examples of which include regret, Quadratic loss function as part of a least squares technique for continuous blood pressure parameter estimation, cross-entropy loss for threshold-based correction activation decisions, custom loss functions that incorporate physiological constraints specific to pulse propagation and vascular mechanics, mean absolute error for blood pressure accuracy optimization, and so forth.

Configuration of the training data is usable to support a variety of usage scenarios in multiparameter cuffless blood pressure monitoring. For example, the machine learning model can be trained to detect specific patterns in pulse propagation time 328 variations that correlate with temperature-induced vasoconstriction or vasodilation, identify hydration-related changes in arterial stiffness that affect pulse wave velocity, recognize perfusion variations that influence peripheral circulation and pulse timing, detect muscle activation patterns that impact arterial wall properties, or establish complex relationships between multiple correction parameters 316 that collectively affect blood pressure measurement accuracy. The models can be configured to operate with threshold-based correction activation to conserve computational resources while providing accurate corrections when correction parameters 316 exceed predetermined ranges. The models can further be reconfigured for continuous correction integration when enhanced accuracy is required across various physiological conditions. This adaptive approach enables efficient use of computational resources devoted to machine learning processes while ensuring comprehensive correction capabilities are available, using the physiological timing data 314 and correction parameters 316 collected during continuous monitoring periods.

It should be understood that many variations are possible based on the disclosure herein. Although features and elements are described above in particular combinations, each feature or element is usable alone without the other features and elements or in various combinations with or without other features and elements.

What is claimed is:

1. A wearable device for cuffless blood pressure monitoring comprising:
a sensor arrangement to collect physiological timing data indicative of a pulse propagation time along a cardiovascular pathway;
a correction sensor configured to measure a correction parameter that impacts the pulse propagation time independent of a corresponding change to blood pressure; and
a processor configured to:
process the physiological timing data to determine the pulse propagation time; and
present a blood pressure measurement generated based on the pulse propagation time and measurements of the correction parameter.

2. The wearable device of claim 1, wherein the sensor arrangement includes a proximal sensor positioned at a proximal location along the cardiovascular pathway configured to detect a first cardiovascular signal as part of the physiological timing data, and a distal sensor positioned at a distal location along the cardiovascular pathway configured to detect a second cardiovascular signal as part of the physiological timing data that corresponds to the first cardiovascular signal.

3. The wearable device of claim 2, wherein the proximal sensor includes one or more of a proximal electrocardiogram sensor, a proximal bioimpedance sensor, or a proximal photoplethysmography sensor, the distal sensor includes one or more of a distal photoplethysmography sensor, a distal pressure sensor, or a distal bioimpedance sensor, and the pulse propagation time includes one or more of a pulse transit time (PTT) or a pulse arrival time (PAT).

4. The wearable device of claim 1, wherein the correction sensor includes one or more of a temperature sensor or a heat flux sensor configured to measure a skin temperature adjacent to the sensor arrangement as the correction parameter, and the processor is further configured to generate the blood pressure measurement based on the skin temperature to account for an impact of vasomotion on the pulse propagation time.

5. The wearable device of claim 1, wherein the correction sensor includes one or more of a temperature sensor or a heat flux sensor configured to measure a body temperature as the correction parameter, and the processor is further configured to generate the blood pressure measurement based on the body temperature to account for systemic cardiovascular effects on the pulse propagation time.

6. The wearable device of claim 1, wherein the correction sensor includes a bioimpedance sensor configured to measure a hydration level as the correction parameter, and the processor is further configured to generate the blood pressure measurement based on the hydration level to account for an impact of arterial stiffness on the pulse propagation time.

7. The wearable device of claim 1, wherein the correction sensor includes a photoplethysmography (PPG) sensor configured to collect PPG data, the processor further configured to:
calculate a perfusion index as the correction parameter by determining a ratio of pulsatile blood flow to non-pulsatile blood flow based on the PPG data; and
generate the blood pressure measurement based on the perfusion index to account for an impact of peripheral circulation on the pulse propagation time.

8. The wearable device of claim 1, wherein the correction sensor includes an electromyography sensor configured to measure muscle activation as the correction parameter by detecting electrical signals associated with smooth muscle tissue, the processor further configured to generate the blood pressure measurement based on the muscle activation to account for arterial wall contractility on the pulse propagation time.

9. The wearable device of claim 1, wherein the processor is further configured to generate the blood pressure measurement by calculating an initial blood pressure measurement based on the pulse propagation time and applying a correction factor to the initial blood pressure measurement based on the correction parameter responsive to a detection that the correction parameter exceeds a threshold.

10. The wearable device of claim 1, wherein the processor is further configured to determine a systolic blood pressure value and a diastolic blood pressure value to include as part of the blood pressure measurement based on one or more characteristics of the pulse propagation time and the correction parameter.

11. A method implemented by a processing device, the method comprising:
receiving physiological timing data indicative of a pulse propagation time along a cardiovascular pathway and measurements of a correction parameter that impacts the pulse propagation time independent of a corresponding change to blood pressure;
processing the physiological timing data to determine the pulse propagation time based on temporal differences between corresponding points in the physiological timing data;
generating a blood pressure measurement based on the pulse propagation time and the measurements of the correction parameter; and
outputting the blood pressure measurement.

12. The method of claim 11, wherein the correction parameter includes at least one of body temperature, skin temperature, perfusion index, hydration level, or muscle activation.

13. The method of claim 11, wherein the physiological timing data includes a first physiological signal and a second physiological signal, and the processing the physiological timing data includes determining one or more of a pulse transit time (PTT) or a pulse arrival time (PAT) as the pulse propagation time based on the first physiological signal and the second physiological signal.

14. The method of claim 13, wherein the first physiological signal includes an electrocardiogram (ECG) signal and the second physiological signal includes a photoplethysmography (PPG) signal.

15. The method of claim 11, wherein generating the blood pressure measurement includes generating an initial blood pressure measurement based on the pulse propagation time and adjusting the initial blood pressure measurement based on the measurements of the correction parameter.

16. The method of claim 11, wherein generating the blood pressure measurement includes applying a correction factor to the blood pressure measurement based on the measurements of the correction parameter responsive to a detection that the correction parameter exceeds a threshold.

17. A system for cuffless blood pressure monitoring comprising:
a sensor arrangement to collect physiological timing data indicative of a pulse propagation time along a cardiovascular pathway;
one or more correction sensors configured to measure one or more correction parameters that impact the pulse propagation time independent of corresponding changes to blood pressure; and
a processor configured to:
process the physiological timing data to determine the pulse propagation time;
generate a blood pressure measurement based on the pulse propagation time and measurements of the one or more correction parameters; and
cause output of the blood pressure measurement.

18. The system of claim 17, wherein the sensor arrangement includes a first sensor configured to measure a first physiological signal via contact with a skin surface of a user and a second sensor configured to measure a second physiological signal via contact with the skin surface of the user, and the processor is further configured to generate one or more of a pulse transit time (PTT) or a pulse arrival time (PAT) as the pulse propagation time based on the first physiological signal and the second physiological signal.

19. The system of claim 17, wherein the measurements of the one or more correction parameters include measurements of at least two of body temperature, skin temperature, perfusion index, hydration level, or muscle activation, and the processor is further configured to implement a weighting scheme to apply weights to the measurements of the one or more correction parameters to generate the blood pressure measurement.

20. The system of claim 17, wherein the processor is configured to implement one or more of a blood pressure calibration algorithm, a machine learning model trained to calibrate blood pressure readings based on correction parameters, or a calibration curve to generate the blood pressure measurement.
